(19) United States
(12) Patent Application Publication
(10) Pub. No.: US 2021/0121136 A1
(43) Pub. Date: Apr. 29, 2021

(54) Screenless Wristband with Virtual Display and Edge Machine Learning

(71) Applicant: Google LLC, Mountain View, CA (US)

(72) Inventors: Kelly Elizabeth Dobson, Mountain View, CA (US); Daniel Mark Kaufman, Redwood City, CA (US)

(21) Appl. No.: 17/082,943

(22) Filed: Oct. 28, 2020

Related U.S. Application Data
(60) Provisional application No. 62/927,123, filed on Oct. 28, 2019.

(51) Int. Cl. A61B 5/00; A61B 5/053

(52) U.S. Cl. CPC A61B 5/7435; A61B 5/7275; A61B 5/681; A61B 5/0531; A61B 5/7267

(57) ABSTRACT

A wearable device includes one or more sensors configured to generate data associated with one or more physiological characteristics of a user of the wearable device and one or more control circuits configured to obtain the data associated with the one or more physiological characteristics of the user and transmit the data to a remote computing device in response to detecting a proximity event associated with the wearable device and the remote computing device.

### RELATED APPLICATIONS

This application is based on and claims priority to U.S. Provisional Patent Application No. 62/927,123, titled “Screenless Wristband with Virtual Display and Edge Machine Learning,” filed on Oct. 28, 2019, which is hereby incorporated by reference herein in its entirety.

### FIELD

The present disclosure relates generally to wearable devices including sensors for measuring physiological responses associated with users of the wearable devices.

### BACKGROUND

Wearable devices integrate electronics into a garment, accessory, container or other article worn or carried by a user. Many wearable devices include various types of sensors integrated within the wearable device to measure attributes associated with a user of the wearable device. By way of example, wearable devices may include heart-rate sensors that measure a heart-rate of a user and motion sensors that measure distances, velocities, steps or other movements associated with a user using accelerometers, gyroscopes, etc. An electrocardiography sensor, for instance, can measure electrical signals (e.g., a voltage potential) associated with the cardiac system of a user to determine a heart rate. A photoplethysmography or other optical-based sensor can measure blood volume to determine heart rate.

### SUMMARY

Aspects and advantages of embodiments of the present disclosure will be set forth in part in the following description, or may be learned from the description, or may be learned through practice of the embodiments.

One example aspect of the present disclosure is directed to a wearable device including one or more sensors configured to generate data associated with one or more physiological characteristics of a user of the wearable device and one or more control circuits configured to obtain the data associated with the one or more physiological characteristics of the user and transmit the data to a remote computing device in response to detecting a proximity event associated with the wearable device and the remote computing device.

Another example aspect of the present disclosure is directed to a user computing device including one or more processors and one or more non-transitory, computer-readable media that store instructions that when executed by the one or more processors cause the one or more processors to perform operations. The operations include determining that a proximity event has occurred between the user computing device and a wearable device including one or more sensors configured to generate data associated with one or more physiological characteristics of a user of the wearable device, receiving, in response to determining that the proximity event has occurred, the data associated with the one or more physiological characteristics of the user, establishing a virtual display connection between the user computing device and the wearable computing device, and generating display data for a graphical user interface including a virtual display associated with the wearable device at the user computing device.

Yet another example aspect of the present disclosure is directed to a wearable device including one or more sensors configured to generate sensor data associated with a user, one or more processors, and one or more non-transitory, computer-readable media that store instructions that when executed by the one or more processors cause the one or more processors to perform operations. The operations include obtaining the sensor data, inputting at least a portion of the sensor data into one or more machine-learned models configured to generate physiological predictions, receiving data indicative of a first physiological prediction from the one or more machine-learned models in response to the at least a portion of the sensor data, generating at least one user notification based at least in part on the physiological prediction, receiving a user confirmation input from the user of the wearable device in association with the physiological prediction, and modifying the one or more machine-learned models based at least in part on the user confirmation input.

Other example aspects of the present disclosure are directed to systems, apparatus, computer program products (such as tangible, non-transitory computer-readable media but also such as software which is downloadable over a communications network without necessarily being stored in non-transitory form), user interfaces, memory devices, and electronic devices for providing map data for display in user interfaces.

These and other features, aspects and advantages of various embodiments will become better understood with reference to the following description and appended claims. The accompanying drawings, which are incorporated in and constitute a part of this specification, illustrate embodiments of the present disclosure and, together with the description, serve to explain the related principles.

### BRIEF DESCRIPTION OF THE DRAWINGS

Detailed discussion of embodiments directed to one of ordinary skill in the art are set forth in the specification, which makes reference to the appended figures, in which:

FIG. 1A-C are perspective views depicting wearable devices including one or more sensors in accordance with example embodiments of the present disclosure.

FIG. 2 depicts a block diagram of a wearable device within an example computing environment in accordance with example embodiments of the present disclosure.

FIG. 3A depicts a block diagram of a wearable device in accordance with example embodiments of the present disclosure.

FIG. 3B depicts a block diagram of a wearable device in accordance with example embodiments of the present disclosure.

FIG. 3C depicts a block diagram of a wearable device in accordance with example embodiments of the present disclosure.

FIG. 4 depicts a remote computing device displaying a graphical user interface associated with a wearable device in accordance with example embodiments of the present disclosure.

FIG. 5 illustrates an example of a virtual display provided by a remote computing device and a wristband in accordance with example embodiments of the present disclosure.

FIG. 6A illustrates an example of a user interaction with a wearable device and a remote computing device in accordance with example embodiments of the present disclosure.

FIG. 6B illustrates an example of graphical user interface provided by a remote computing device in accordance with example embodiments of the present disclosure.

FIG. 6C illustrates an example of a wearable device providing a user notification in accordance with example embodiments of the present disclosure.

FIG. 6D illustrates an example of a user interaction with a wearable device in accordance with example embodiments of the present disclosure.

FIG. 6E illustrates an example of a user interaction with a remote computing device in accordance with example embodiments of the present disclosure.

FIG. 7 is a flowchart depicting an example process in accordance with example embodiments of the present disclosure.

FIG. 8A illustrates an example of a user interaction with a wearable device and a remote computing device in accordance with example embodiments of the present disclosure.

FIG. 8B illustrates an example of graphical user interface provided by a remote computing device in accordance with example embodiments of the present disclosure.

FIG. 8C illustrates an example of a wearable device providing a user notification in accordance with example embodiments of the present disclosure.

FIG. 8D illustrates an example of a user interaction with a wearable device in accordance with example embodiments of the present disclosure.

FIG. 8E illustrates an example of a user interaction with a remote computing device in accordance with example embodiments of the present disclosure.

FIG. 8F illustrates an example of a wearable device 202 using generated sensor data to train a one or more machine-learned physiological response prediction models for a user in accordance with example embodiments of the present disclosure.

FIG. 8G illustrates an example of a user confirmation of a physiological response prediction provided by the wearable device in accordance with example embodiments of the present disclosure.

FIG. 8H illustrates an example of a virtual display provided by a remote computing device and a wristband in accordance with example embodiments of the present disclosure.

FIG. 9 is a flowchart describing an example process in accordance with example embodiments of the present disclosure.

FIG. 10 is a flowchart describing an example process in accordance with example embodiments of the present disclosure.

FIG. 11 is a flowchart describing an example process in accordance with example embodiments of the present disclosure.

FIG. 12A illustrates an example of a user interaction with a wearable device and a remote computing device in accordance with example embodiments of the present disclosure.

FIG. 12B illustrates an example of graphical user interface provided by a remote computing device in accordance with example embodiments of the present disclosure.

FIG. 12C illustrates an example of a wearable device providing a user notification in accordance with example embodiments of the present disclosure.

FIG. 12D illustrates an example of a wearable device providing an output in accordance with example embodiments of the present disclosure.

FIG. 12E illustrates an example of a virtual display provided by a a remote computing device and a wristband in accordance with example embodiments of the present disclosure.

FIG. 12F illustrates an example of a user interaction with a remote computing device in accordance with example embodiments of the present disclosure.

FIG. 12G illustrates an example of a user interaction with a remote computing device in accordance with example embodiments of the present disclosure.

FIG. 12H illustrates an example of a wearable device providing an output in accordance with example embodiments of the present disclosure.

FIG. 13 is a flowchart describing an example process in accordance with example embodiments of the present disclosure.

FIG. 14 is a flowchart describing an example process in accordance with example embodiments of the present disclosure.

FIG. 15 is a flowchart describing an example process in accordance with example embodiments of the present disclosure.

FIG. 16 depicts a block diagram of an example computing environment including a wearable device in accordance with example embodiments of the present disclosure.

FIG. 17A depicts a block diagram of an example computing device in accordance with example embodiments of the present disclosure.

FIG. 17B depicts a block diagram of an example computing device in accordance with example embodiments of the present disclosure.

FIG. 18 depicts a block diagram of an example machine-learned system including one or more machine-learned models in accordance with example embodiments of the present disclosure.

FIG. 19 depicts a block diagram of an example machine-learned system including machine-learned models in accordance with example embodiments of the present disclosure.

### DETAILED DESCRIPTION

Reference now will be made in detail to embodiments, one or more examples of which are illustrated in the drawings. Each example is provided by way of explanation of the embodiments, not limitation of the present disclosure. In fact, it will be apparent to those skilled in the art that various modifications and variations can be made to the embodiments without departing from the scope or spirit of the present disclosure. For instance, features illustrated or described as part of one embodiment can be used with another embodiment to yield a still further embodiment. Thus, it is intended that aspects of the present disclosure cover such modifications and variations.

Generally, the present disclosure is directed to wearable devices that include sensor systems configured to measure physiological characteristics associated with users of the wearable devices. More particularly, systems and methods in accordance with example embodiments are provided for measuring physiological characteristics and automatically generating displays at remote computing devices based on data indicative of the physiological characteristics. By way of example, a screenless wristband may include one or more sensors that are configured to measure physiological characteristics associated with a user and generate sensor data indicative of the physiological characteristics. A remote computing device such as a user's smart phone may automatically generate one or more displays indicative of the physiological characteristics of a user in response to detecting a proximity event between the wearable device and remote computing device. The proximity event may be detected by the remote computing device and/or the user smart phone. By way of example, a wearable device and remote computing device may be automatically and communicatively coupled using a Bluetooth, near field communication, UWB, or other suitable connection. By way of example, the wristband and a corresponding smartphone app (e.g., device manager) can be configured such that, if a user brings their smartphone within a threshold distance of the wristband, the smartphone display will detect a proximity event and immediately and automatically be triggered to display information content that corresponds to the readings taken by the wristband (e.g., blood pressure, heart rate, etc).

In many traditional examples, wearable devices are equipped with high-definition or other types of displays in order to provide a user with information regarding sensor data or other characteristics associated with the user. In accordance with example embodiments of the present disclosure, however, a screenless wristband is provided such that a small form factor device can be realized. Nevertheless, the wristband in combination with a remote computing device such as a user smart phone can implement a virtual display to provide a seamless interface whereby a user can understand the sensor data associated physiological responses.

In accordance with some examples, a wearable device such as a smart wristband may include one or more machine learned models that can be trained locally at the wearable device using sensor data generated by the wearable device. In some examples, a user can provide an input indicating a particular physiological response or state of the user. For instance, a user may indicate that they are stressed by providing input to the wearable device. In response, the wearable device can log sensor data associated with the identified time. The sensor data can be annotated to indicate that it corresponds to a stress event. The annotated sensor data can be used to generate training data that is used to train the machine learned model at the wearable device. In other examples, one or more machine learned models may generate a prediction such as a predicted physiological response. A user can provide user confirmation input to confirm that the physiological response prediction was correct or to indicate that the physiological response prediction was incorrect. The user confirmation input and sensor data can be used to generate training data that is further used to train one or more machine learned models.

In accordance with example, embodiments, a virtual display provided by a remote computing device can be updated based on the relative movement between the remote computing device and the wearable device. For example, a the user moves the remote computing device (e.g., display of smartphone) in physical relation to the wearable device (e.g., band) the display can be smoothly transitioned to different views of the data and data derived experiences that an application associated with the wearable device is serving. This awareness of movement and pose in relation to the band can be achieved by several methods. Example methods include but are not limited to using an image capture device such as a camera of the remote computing device and on-device image processing on the remote computing device to capture images of the wearable device worn by the user (e.g., on the wearer's arm) and calculate the phone's relative distance and pose. In another example, EMF modelling and real-time analysis, IR range finding, or other methods may be used. In accordance with some examples, an image capture device can be used so that an augmented reality layer can be provided. The graphical user interface can include an image presented to the user where some of the image is a photographic image from the camera and some is a view of representations of data. The graphical user interface can present the image in a zoom in and out level of detail and selection, as if the wearable device itself opened up multiple spatial/physiological/contextual dimensions. These dimensions can be provided and with the remote computing device and the wearable device these dimensions can be navigated in real time by the user seamlessly.

With reference now to the figures, example aspects of the present disclosure will be discussed in greater detail.

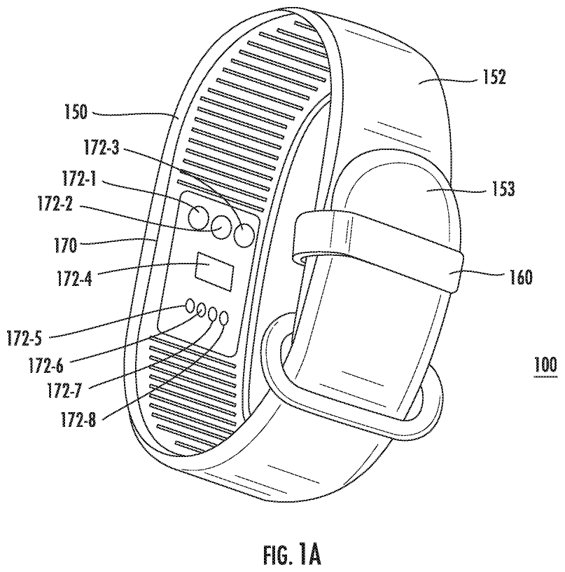

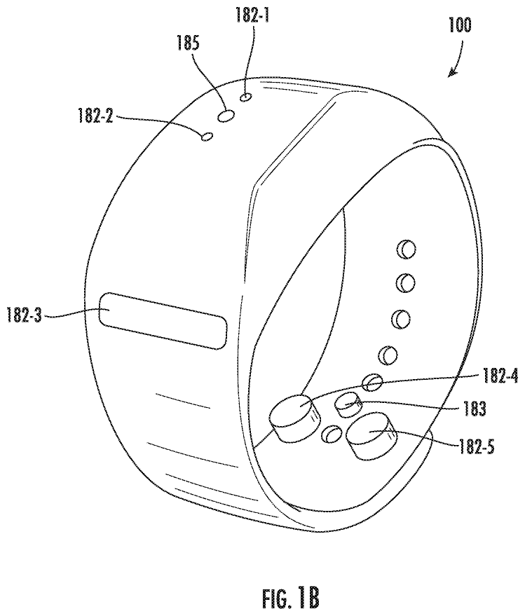

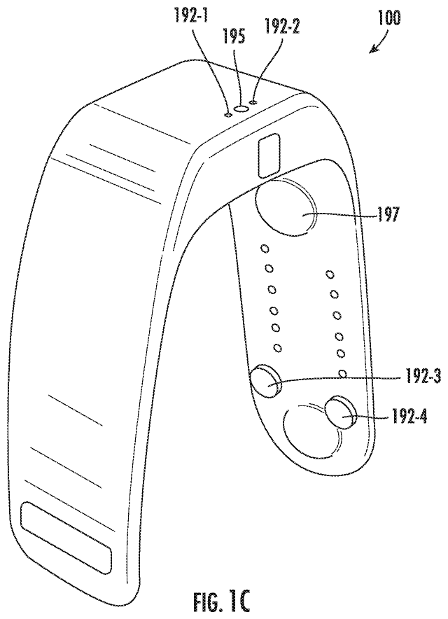

FIGS. 1A-1C are perspective views depicting example implementations of a wearable device 100 including one or more sensors in accordance with example embodiments of the present disclosure. Wearable device 100 includes an attachment member 150 which in various examples may take the form of a band or a strap configured to wrap around a wrist, ankle, or other body part of the user when wearable device 100 is worn by the user. Attachment member 150 can include a first end 152 and a second end 153 that are joined using a fastener 160 such as a clasp or magnet, or other fastener to form a secure attachment when worn, however many other designs may be used. The strap or other attachment member can be formed from a material such as rubber, nylon, plastic, metal, or any other type of material suitable to send and receive visual, audible, and/or haptic responses. Notably, however, wearable device 100 may take any type or form. For example, rather than being a strap, attachment member 150 may resemble a circular or square piece of material (e.g., rubber or nylon) that can be attached to the plurality of sensors and substrate material of a wearable device such as a garment. Due to the small form factor and integrated nature of various electrodes, wearable device 100 can provide a non-obtrusive and effective sensor system for measuring various physiological characteristics or responses associated with the user.

Wearable device 100 includes a sensor system 170 including multiple sensor electrodes 172-1 to 172-8. Sensor system 170 can include one or more sensors configured to detect various physiological responses of a user. For instance, sensor system 170 can include an electrodermal activity sensor (EDA), a photoplethysmogram (PPG) sensor, a skin temperature sensor, and/or an inertial measurement unit (IMU). Additionally or alternatively, sensor system can include an electrocardiogram (ECG) sensor, an ambient temperature sensor (ATS), a humidity sensor, a sound sensor such as a microphone (e.g., ultrasonic), an ambient light sensor (ALS), a barometric pressure sensor (e.g., barometer)

Sensor electrodes 172-1 to 172-8 are positioned on an inner surface of the attachment member 150 (e.g., band) where they can contact the skin of a user at a desired location of the user's body when worn. By way of example, the sensor system 170 can include a lower surface 142 that is physically coupled to the attachment member 150 such as the band or strap forming all or part of the wearable device, and an upper surface that is configured to contact the surface of the user's skin. The lower surface of the sensor system 170 can be directly coupled to the attachment member 150 of the wearable device in example embodiments. The sensor system 170 can be fastened (permanently or removably) to the attachment member, glued to the attachment member, or otherwise physically coupled to the attachment member. In some examples, the lower surface of the sensor system 170 can be physically coupled to the attachment member 150 or other portion of the wearable device 100 via one or more intervening members. In some examples, portions of sensor system 170 may be integrated directly within attachment member 150.

Individual sensors of sensor system 170 may include or otherwise be in communication with sensor electrodes 172-1-172-8 in order to measure physiological responses associated with a user. For example, an electrodermal activity (EDA) sensor can be configured to measure conductance or resistance associated with the skin of a user to determine EDA associated with a user of the wearable device 100. As another example, sensor system 170 can include a PPG sensor including one or more sensor electrodes 172 configured to measure the blood volume changes associated with the microvascular tissue of the user. As another example, sensor system 170 can include a skin temperature sensor including one or more sensor electrodes 172 configured to measure the temperature of the user's skin. As another example, sensor system 170 can include an ECG sensor including one or more sensor electrodes 172 configured to measure the user's heart rate.

In some embodiments, wearable device 100 can include one or more input devices and/or one or more output devices. An input device such as a touch input device can be utilized to enable user to provide input to the wearable device. An output device can be configured to provide a haptic response, a tactical response, an audio response, a visual response, or some combination thereof. Output devices may include visual output devices, such as one or more light-emitting diodes (LEDs), audio output devices such as one or more speakers, one or more tactile output devices, and/or one or more haptic output devices. In some examples, the one or more output devices are formed as part of the wearable device, although this is not required. In one example, an output device can include one or more LEDs configured to provide different types of output signals. For example, the one or more LEDs can be configured to generate patterns of light, such as by controlling the order and/or timing of individual LED activations based on physiological activity. Other lights and techniques may be used to generate visual patterns including circular patterns. In some examples, one or more LEDs may produce different colored light to provide different types of visual indications. Output devices may include a haptic or tactile output device that provides different types of output signals in the form of different vibrations and/or vibration patterns. In yet another example, output devices may include a haptic output device such as may tighten or loosen a wearable device with respect to a user. For example, a clamp, clasp, cuff, pleat, pleat actuator, band (e.g., contraction band), or other device may be used to adjust the fit of a wearable device on a user (e.g., tighten and/or loosen). In some examples, wearable device 100 may include a simple output device that is configured to provide a visual output based on a level of one or more signals detected by the sensor system. By way of example, wearable device may include one or more light emitting diodes. In other examples, however, a wearable device may include processing circuitry configured to process one or more sensor signals to provide enhanced interpretive data associated with a user's physiological activity.

It is noted that while a human being is typically referred to herein, a wearable device as described may be used to measure electrodermal activity associated with other living beings such as dogs, cats, or other animals in accordance with example embodiments of the disclosed technology.

FIG. 1B depicts another example of a wearable device 100 including sensor electrodes 182-1, 182-2, 182-3, 182-4, and 182-5, fastening member 183 and an output device 185 (e.g., LED). FIG. 1C depicts an example of wearable device 100 including sensor electrodes 192-1, 192-2, 192-3, 192-4, and 192-5, an output device 195 (e.g., LED), and a haptic output device 197.

FIG. 2 depicts a block diagram of a wearable device 202 within an example computing environment 200 in accordance with example embodiments of the present disclosure. FIG. 2 depicts a user 220 wearing a wearable device 202. In this example, wearable device 202 is worn around the user's wrist using an attachment number such that the sensors 204 of the wearable device are in contact with the skin of the user. By way of example, wearable device 202 may be a smartwatch, a wristband, a fitness tracker, or other wearable device. It is noted that while FIG. 2 depicts an example of a wearable device worn around the user's wrist, wearable devices of any form can be utilized in accordance with embodiments of the present disclosure. For instance, sensors 204 can be integrated into wearable devices that are coupled to a user in other manners, such as into garments that are worn or accessories that are carried.

FIG. 2 illustrates an example environment 200 that includes a wearable device 202 that is capable of communication with one or more remote computing devices 260 over one or more networks 250. Wearable device 202 can include one or more sensors 204, sensing circuitry 206, processing circuitry 210, input/output device(s) 214 (e.g., speakers, LEDs, microphones, touch sensors), power source 208 (e.g., battery), memory 212 (RAM and/or ROM), and/or a network interface 216 (e.g., Bluetooth, WiFi, USB). Sensing circuitry may be a part of sensors 204 or separate from the sensors 204. Wearable device 202 is one example of a wearable device as described herein. It will be appreciated while specific components are depicted in FIG. 2, additional or fewer components may be included in a wearable device in accordance with example embodiments of the present disclosure.

In environment 200, the electronic components contained within the wearable device 202 include sensing circuitry 206 that is coupled to a plurality of sensors 204. Sensing circuitry 206 can include various components such as amplifiers, filters, charging circuits, sense nodes, and the like that are configured to sense one or more physical or physiological characteristics or responses of a user via the plurality of sensors 204. Power source 208 may be coupled, via one or more interfaces to provide power to the various components of the wearable device, and may be implemented as a small battery in some examples. Power source 208 may be coupled to sensing circuitry 206 to provide power to sensing circuitry 206 to enable the detection and measurement of a user's physiological and physical characteristics. Power source 208 can be removable or embedded within a wearable device in example embodiments. Sensing circuitry 206 can be implemented as voltage sensing circuitry, current sensing circuitry, capacitive sensing circuitry, resistive sensing circuitry, etc.

By way of example, sensing circuitry 206 can cause a current flow between EDA electrodes (e.g., an inner electrode and an outer electrode) through one or more layers of a user's skin in order to measure an electrical characteristic associated with the user. In some examples, sensing circuitry 206 can generate an electrodermal activity signal that is representative of one or more electrical characteristics associated with a user of the wearable device. In some examples, an amplitude or other measure associated with the EDA signal can be representative of sympathetic nervous system activity of a user. The EDA signal can include or otherwise be indicative of a measurement of conductance or resistance associated with the user's skin as determined using a circuit formed with the integrated electrode pair. By way of example, the sensing circuitry and an integrated electrode pair can induce a current through one or more dermal layers of a user's skin. The current can be passed from one electrode into the user's skin via an electrical connection facilitated by the user's perspiration or other fluid. The current can then pass through one or more dermal layers of the user's skin and out of the skin and into the other electrode via perspiration between the other electrode and the user's skin. The sensing circuitry can measure a buildup and excretion of perspiration from eccrine sudoriferous glands as an indicator of sympathetic nervous system activity in some instances. For example, the sensing circuitry may utilize current sensing to determine an amount of current flow between the concentric electrodes through the user's skin. The amount of current may be indicative of electrodermal activity. The wearable device can provide an output based on the measured current in some examples.

Processing circuitry 210 can include one or more electric circuits that comprise one or more processors such as one or more microprocessors. Memory 212 can include (e.g., store, and/or the like) instructions. When executed by processing circuitry 210, instructions stored in memory 212 can cause processing circuitry 210 to perform one or more operations, functions, and/or the like described herein. Processing circuitry can analyze the data from the plurality of sensors or other physiological or physical responses associated with the user of the wearable device in order to determine data indicative of the stress a user is under. By way of example, processing circuitry 210 can generate data indicative of metrics, heuristics, trends, predictions, or other measurements associated with a user's physiological or physical responses.

Wearable device 202 may include one or more input/output devices 214. An input device such as a touch input device can be utilized to enable user to provide input to the wearable device. An output device such as a touch device can be utilized to enable user to view the output from the wearable device. An output device can be configured to provide a haptic response, a tactical response, an audio response, a visual response, or some combination thereof. Output devices may include visual output devices, such as one or more light-emitting diodes (LEDs), audio output devices such as one or more speakers, one or more tactile output devices, and/or one or more haptic output devices. In some examples, the one or more output devices are formed as part of the wearable device, although this is not required. In one example, an output device can include one or more devices configured to provide different types of haptic output signals. For example, the one or more haptic devices can be configured to generate specific output signals in the form of different vibrations and/or vibration patterns based on the user's stress, and the user's physical and physiological responses. In another example, output devices may include a haptic output device such as may tighten or loosen a wearable device with respect to a user. For example, a clamp, clasp, cuff, pleat, pleat actuator, band (e.g., contraction band), or other device may be used to adjust the fit of a wearable device on a user (e.g., tighten and/or loosen). In one example, an output device can include one or more LEDs configured to provide different types of output signals. For example, the one or more LEDs can be configured to generate patterns of light, such as by controlling the order and/or timing of individual LED activations based on the user's stress, and/or other user physical and physiological responses. Other lights and techniques may be used to generate visual patterns including circular patterns. In some examples, one or more LEDs may produce different colored light to provide different types of visual indications.

Network interface 216 can enable wearable device 202 to communicate with one or more computing devices 260. By way of example and not limitation, network interfaces 216 may communicate data over a local-area-network (LAN), a wireless local-area-network (WLAN), a personal-area-network (PAN) (e.g., Bluetooth™), a wide-area-network (WAN), an intranet, the Internet, a peer-to-peer network, point-to-point network, a mesh network, and the like. Network interface 216 can be a wired and/or wireless network interface.

By way of example, wearable device 202 may transmit data indicative of a user's physical and physiological characteristics to one or more remote computing devices in example embodiments. As described herein, a proximity event may be detected by a wearable device and/or a remote computing device. For instance, in response to detecting that a position of the remote computing device relative to the wearable device satisfies one or more thresholds (e.g., proximity constraints), the wearable device can automatically transmit data indicative of physical and/or physiological characteristics or responses detected by one or more sensors 204 of the wearable device. The data may include raw sensor data as generated by one or more sensors 204 in example embodiments. In some examples, the data may include data derived from or otherwise based at least in part on the sensor data. For instance, the data may include detections of predetermined physiological activity, data indicative of physical and physiological characteristics or responses, or other data associated with the user. The data may be communicated, via network interface 216, to a remote computing device 260 via network 250. In some examples, one or more outputs of sensing circuitry 206 are received by processing circuitry 221 (e.g., microprocessor) The processing circuitry may analyze the output of the sensors (e.g., an ECG signal) to determine data associated with a user's physical and physiological responses. The data and/or one or more control signals may be communicated to a computing device 260 (e.g., a smart phone, server, cloud computing infrastructure, etc.) via the network interface 216 to cause the computing device to initiate a particular functionality. Generally, network interfaces 216 are configured to communicate data, such as ECG data, over wired, wireless, or optical networks to computing devices, however, any suitable connection may be used.

In some examples, the internal electronics of the wearable device 202 can include a flexible printed circuit board (PCB). The printed circuit board can include a set of contact pads for attaching to the integrated electrode pair 804. In some examples, one or more of sensing circuitry 206, processing circuitry 210, input/output devices 214, memory 212, power source 208, and network interface 216 can be integrated on the flexible PCB.

Wearable device 202 can include various other types of electronics, such as additional sensors (e.g., capacitive touch sensors, microphones, accelerometers, ambient temperature sensor, barometer, ECG, EDA, PPG), output devices (e.g., LEDs, speakers, or haptic devices), electrical circuitry, and so forth. The various electronics depicted within wearable device 202 may be physically and permanently embedded within wearable device 202 in example embodiments. In some examples, one or more components may be removably coupled to the wearable device 202. By way of example, a removable power source 208 may be included in example embodiments.

While wearable device 202 is illustrated and described as including specific electronic components, it will be appreciated that wearable devices may be configured in a variety of different ways. For example, in some cases, electronic components described as being contained within a wearable device may at least be partially implemented at another computing device, and vice versa. Furthermore, wearable device 202 may include electronic components other that those illustrated in FIG. 2, such as sensors, light sources (e.g., LED's), displays, speakers, and so forth.

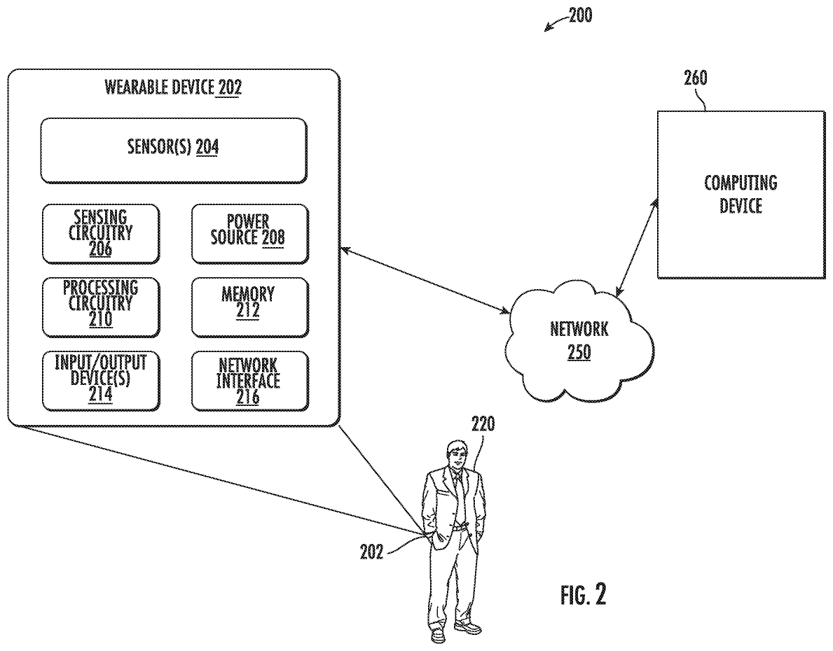

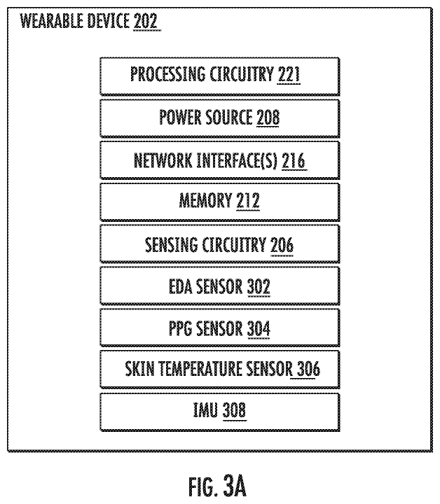

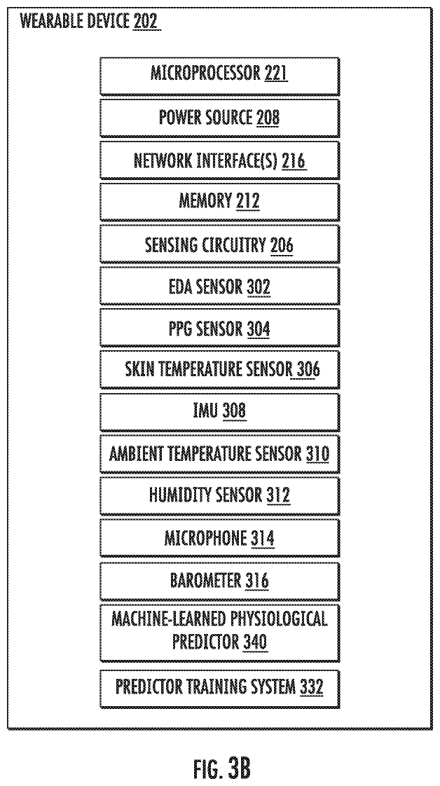

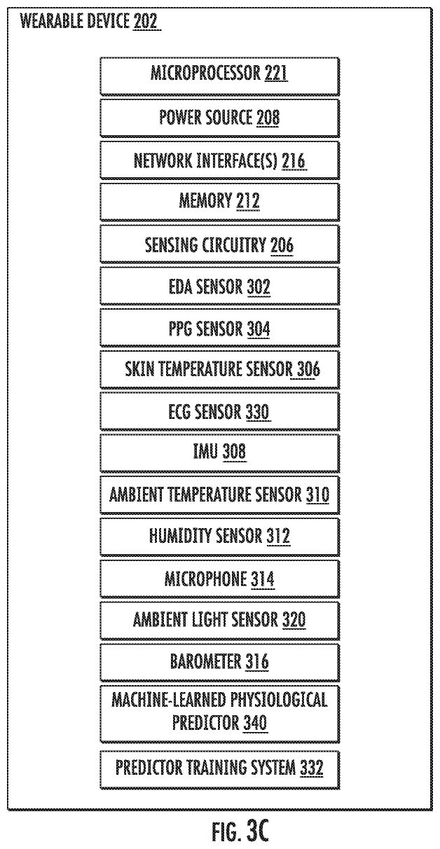

FIG. 3A is a block diagram depicting an example wearable device 202 in accordance with example embodiments of the present disclosure. Wearable device 202 includes processing circuitry 221 (e.g, microprocessor), power source 208, network interface(s) 216, memory 212, sensing circuitry 206 communicatively coupled to a plurality of sensors 204 including but not limited to an electrodermal activity sensor (EDA) 302, photoplethysmogram (PPG) 304, skin temperature sensor 306, and IMU 308. Wearable device may generate a visual, audible, and/or haptic output based on the user's physical and physiological responses based on the data from the plurality of sensors 204. An electrodermal activity (EDA) sensor 302 can be configured to measure conductance or resistance associated with the skin of a user to determine EDA associated with a user of the wearable device 100.

Photoplethysmogram (PPG) sensor 304 can generate sensor data indicative of changes in blood volume in the microvascular tissue of a user. The PPG sensor may generate one or more outputs describing the changes in the blood volume in a user's microvascular tissue. PPG sensor 304 can include one or more light emitting diodes and one or more photodiodes. In an example, PPG sensor 304 can include one photodiode. In another embodiment, PPG sensor 304 can include more than one photodiode. Sensing circuitry 206 can cause an LED to illuminate the user's skin in contact with the wearable device 202 and sensing system 170, in order to measure the amount of light reflected to the one or more photodiodes from blood in the microvascular tissue. The amount of light transmitted or reflected is indicative of the change in blood volume.

The ECG 330 can generate sensor data indicative of the electrical activity of the heart using electrodes in contact with the skin. The ECG 330 can comprise one or more electrodes in contact with the skin of a user. The sensing system 170 may comprise one or more electrodes to measure a user's ECG, with one end of each electrode connected to the lower surface of the band of the wearable device and the other in contact with the user's skin.

The skin temperature sensor 306 can generate data indicative of the user's skin temperature. The skin temperature sensor can include one or more thermocouples indicative of the temperature and changes in temperature of a user's skin. The sensing system 170 may include one or more thermocouples to measure a user's skin temperature, with the thermocouple in contact with the user's skin.

The inertial measurement unit(s) (IMU(s)) 308 can generate sensor data indicative of a position, velocity, and/or an acceleration of the interactive object. The IMU(s) 308 may generate one or more outputs describing one or more three-dimensional motions of the wearable device 202. The IMU(s) may be secured to the sensing circuitry 206, for example, with zero degrees of freedom, either removably or irremovably, such that the inertial measurement unit translates and is reoriented as the wearable device 202 is translated and are reoriented. In some embodiments, the inertial measurement unit(s) 308 may include a gyroscope or an accelerometer (e.g., a combination of a gyroscope and an accelerometer), such as a three axis gyroscope or accelerometer configured to sense rotation and acceleration along and about three, generally orthogonal axes. In some embodiments, the inertial measurement unit(s) may include a sensor configured to detect changes in velocity or changes in rotational velocity of the interactive object and an integrator configured to integrate signals from the sensor such that a net movement may be calculated, for instance by a processor of the inertial measurement unit, based on an integrated movement about or along each of a plurality of axes.

FIG. 3BA is a block diagram depicting an example wearable device 202 in accordance with example embodiments of the present disclosure. Wearable device 202 can include processing circutry 221, power source 208, network interface(s) 216, memory 212, sensing circuitry 206 coupled with a plurality of sensors 204 including but not limited to an EDA 302, PPG 304, skin temperature sensor 306, IMU 308, ambient temperature sensor (ATS) 310, humidity sensor 312, microphone 314, and barometer 316. In an example, the wearable device can include a machine-learned physiological predictor 330 configured to predict a user's physiological responses, and a predictor training system 332 configured to train the machine-learned physiological predictor. Wearable device 202 may generate a visual, audible, and/or haptic output based on the user's physical and physiological responses based on the data from the plurality of sensors 204.

FIG. 3C is a block diagram depicting an example wearable device 202 in accordance with example embodiments of the present disclosure. Wearable device 202 can include processing circuitry 221, power source 208, network interface(s) 216, memory 212, sensing circuitry 206 coupled with a plurality of sensors 204 including but not limited to an EDA 302, PPG 304, skin temperature sensor 306, electrocardiogram (ECG) 330, IMU 308, ATS 310, humidity sensor 312, microphone 314, ambient light sensor (ALS) 320, and barometer 316. In an example, the wearable device can include a machine-learned physiological predictor 340 configured to predict a user's physiological responses, and a predictor training system 332 configured to train the machine-learned physiological predictor. Wearable device may generate a visual, audible, and/or haptic output based on the user's physical and physiological responses based on the data from the plurality of sensors 204.

In some examples, an amplitude or other measure associated with a sensor signal (e.g., EDA signal, ECG signal, PPG signal) can be representative of one or more physiological characteristics associated with a user, such as sympathetic nervous system activity of a user. For instance, a sensor signal can include or otherwise be indicative of a measurement of conductance or resistance associated with the user's skin as determined using a circuit formed with an integrated electrode pair. Such signals can be electrical, optical, electro-optical, or other types of signals.

The inertial measurement unit(s) (IMU(s)) 308 can generate sensor data indicative of a position, velocity, and/or an acceleration of the interactive object. The IMU(s) 308 may generate one or more outputs describing one or more three-dimensional motions of the wearable device 202. The IMU(s) may be secured to the sensing circuitry 206, for example, with zero degrees of freedom, either removably or irremovably, such that the inertial measurement unit translates and is reoriented as the wearable device 202 is translated and are reoriented. In some embodiments, the inertial measurement unit(s) 308 may include a gyroscope or an accelerometer (e.g., a combination of a gyroscope and an accelerometer), such as a three axis gyroscope or accelerometer configured to sense rotation and acceleration along and about three, generally orthogonal axes. In some embodiments, the inertial measurement unit(s) may include a sensor configured to detect changes in velocity or changes in rotational velocity of the interactive object and an integrator configured to integrate signals from the sensor such that a net movement may be calculated, for instance by a processor of the inertial measurement unit, based on an integrated movement about or along each of a plurality of axes. In some examples, a full IMU may not be used. For example, a wearable device may include a gyroscope or accelerometer in some examples. Any number gyroscopes and/or accelerometers may be used.

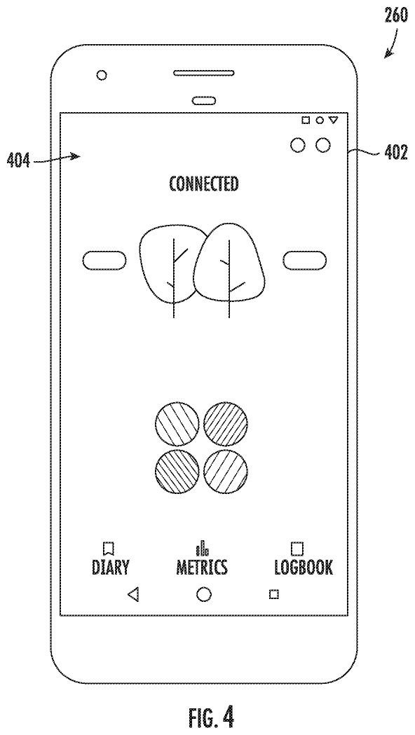

FIG. 4 depicts an example of a remote computing device 260 implemented as a user computing device having a display 402 that provides a graphical user interface 404 associated with a wearable device in accordance with example embodiments of the present disclosure. The user interface 404 provided by the remote computing device displays one or more graphical representations of sensor data that is communicated to the remote computing device from the wearable device. The user interface can provide a display of raw sensor data communicated by the wearable device and/or various data derived from the raw sensor data, such as various analyses of the sensors data. The data derived from the sensor data may be generated by the wearable device and communicated to the remote computing device and/or may be determined by the remote computing device. By way of example, the user interface may display one or more charts that indicate the times the user's EDA signals or PPG signals were over a certain predetermined threshold. In another example, the user interface can display resources that may be useful to the user of the wearable device based on the sensor data and the analyses of the sensor data. For example, the user interface may provide a user with additional information on ways to lower the user's heart rate. In some examples, the user interface may display information regarding patterns of physiological activity associated with a user.

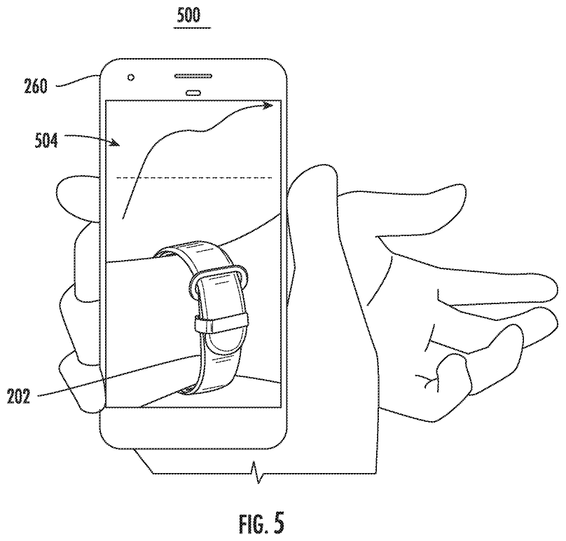

FIG. 5 depicts an example of a remote computing device 260 implemented as a user computing device providing a graphical user interface 504 including a virtual display of sensor data generated by a wearable device in association with a user. A user computing device is one example of a remote computing device 260. According to example aspects of the present disclosure, a relative position of the remote computing device 260 to the wearable device 202 can be determined. The wearable device and/or remote computing device can determine if one or more thresholds (e.g., positional constraints) are satisfied by the relative position. For instance, a positional constraint can specify a threshold distance. If the relative position indicates that the two devices are within the threshold distance, the positional constraint can be satisfied. In another example, a threshold may include a time constraint. For instance, a time constraint can specify a threshold time that the remote computing device 260 is within a threshold distance. Other thresholds may be used such as more precise positioning of the remote computing device to the wearable device. For instance, it can be determined whether the remote computing device is positioned above (e.g., hovered over) and within a predetermined distance of the wearable device 202 in order to determine if one or more thresholds have been satisfied. In yet another example, a positional constraint can include a relative direction of motion between the remote computing device 260 and the wearable device 202. If the one or more thresholds are satisfied, the graphical user interface 504 at the remote computing device 260 can be updated based on the sensor data or other data generated by the wearable device 202 and sent to the computing device. By way of example, the remote computing device can automatically generate the user interface to display sensor data or other data from the wearable device in response to determining that the one or more thresholds are satisfied. In this manner, the remote computing device can provide a seamless virtual display into the insights gathered by the wearable device.

In one example, the wearable device 202 may update the graphical user interface 504 at the remote computing device 260 to enable a virtual display associated with the wearable device 202 based on the sensor data from the wearable device 202. In an example, if the relative position of the remote computing device 260 to the wearable device 202 satisfies the one or more positional constraints, then the wearable device 202 may establish a virtual display connection with the remote computing device, and update the graphical user interface 504 at the remote computing device 260 to enable a virtual display associated with the wearable device 202. In one example, the virtual display on the graphical user interface 504 of the remote computing device 260 may provide a first display including a real-time depiction of the position of the body part on which the wearable device 202 is worn and shape of the wearable device 202 on that body part. For example, if a user hovers the remote computing device 260 (e.g., smartphone) over a smart wristband satisfying the one or more positional constraints, the graphical user interface of the remote computing device 260 may display imagery (e.g., one or more images or videos) captured by one or more image sensors (e.g, cameras) depicting the real-time position of the user's hand and of the smart wristband on the hand of the user. In another example, the virtual display on the graphical user interface may provide a second display including a depiction of sensor data or data derived from the sensor data generated by the wearable device. By way of example, the second display may include representations of raw sensor data, analyses of raw sensor data, predictions based on raw sensor data, etc. In some examples, data may be displayed by projecting the graphical user interface on the surface of the wearable device 202 and/or the user. In another example, the virtual display of the graphical user interface may include a depiction of sensor data or other data on the surface of the user's skin adjacent to the wearable device 202.

In one example, if the relative position of the remote computing device 260 to the wearable device 202 satisfies the one or more positional constraints, the remote computing device 260 may initiate a virtual display of the graphical user interface 504, and update the virtual display based on the sensor data from the wearable device 202.

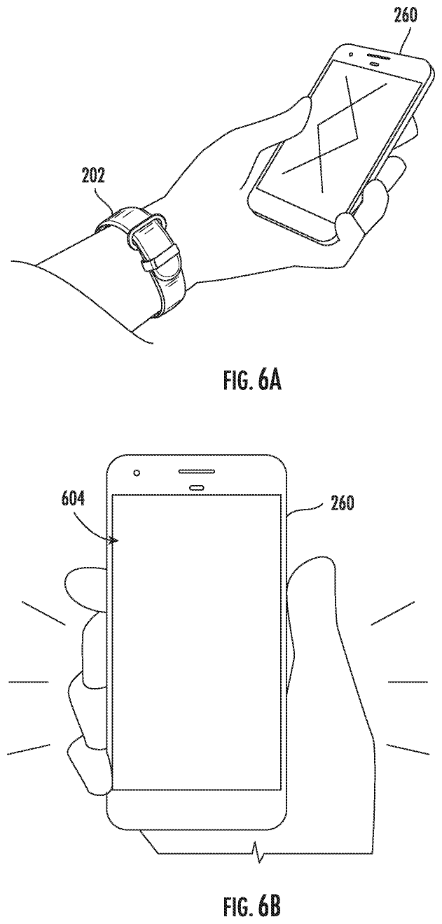

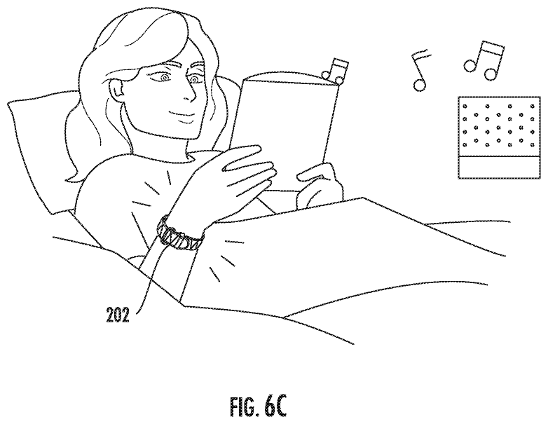

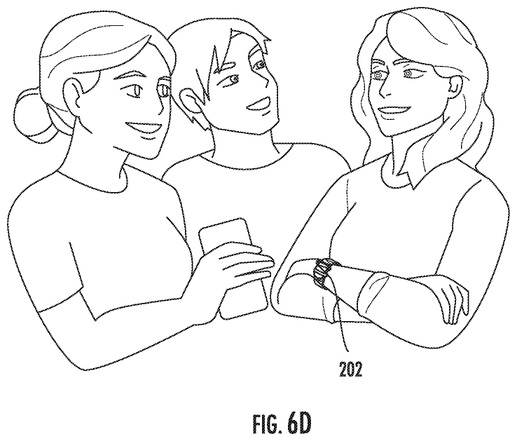

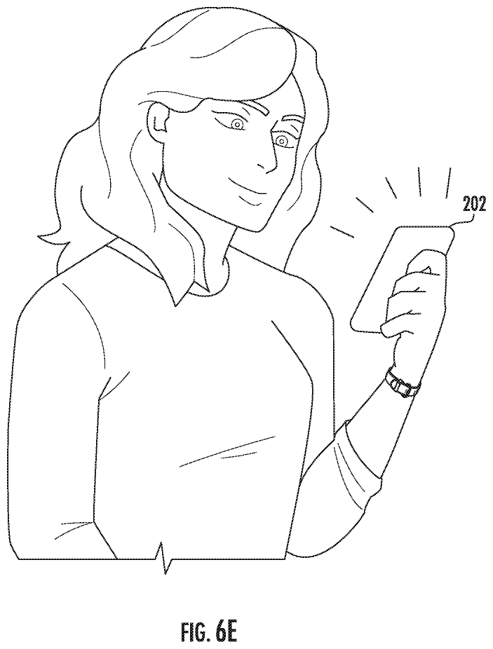

FIGS. 6A-6E are graphical depictions of example user interactions with a wearable device 202 and a remote computing device 260.

FIG. 6A depicts a remote computing device 260 implemented as a user computing device (e.g., user's smartphone) and a wearable device 202 (e.g., smart wristband). A user may set up the wearable device to communicate with the remote computing device. In example embodiments, the wearable device 202 and the remote computing device 260 can be communicatively coupled using an application programming interface that enables the remote computing device 260 and the wearable device 202 to communicate. In some examples, the wearable device can include a wristband manager that can interface with the wristband to provide information to a user (e.g., through a display, audible output, haptic output, etc.) and to facilitate user interaction with the wristband. Additionally or alternatively, remote computing device 260 can include a device manager configured to generate one or more graphical user interfaces that can provide information associated with wearable device 202. By way of example, a device manager at a remote computing device can generate one or more graphical user interfaces that provide graphical depictions of sensor data or data derived from sensor data. As an example, a user can buy a new product to help with stress. It may be a wristband or other wearable device with sensors inside. The user can put it on and pair it with an app on their phone.

FIG. 6B depicts a graphical user interface (GUI) 604 displayed by the remote computing device 260 in accordance with example embodiments of the present disclosure. In various examples, the GUI provides a display including information that informs the user as to the use of the wristband and how it can be used to benefit the user. By way of example, the GUI may provide an indication that the wristband can be used to detect various physiological responses associated with a user and provide information associated with the various physiological responses. The GUI may provide an indication that detecting physiological responses and providing such information may be used to benefit the user. The GUI (e.g., provided by an application such as a device manager) can teach a user to think differently about stress and use it to their benefit.

FIG. 6C depicts an example of a wearable device 202 generating user notifications. The user notifications can be visual, aural, and/or haptic in nature. The user notifications can be generated at periodic intervals or at random intervals. For example, the wearable device 202 may generate vibrations and/or vibration patterns at random times. The user notifications can be provided to remind to the user to think about the beneficial information pertaining to physiological responses that the was displayed by the user's remote computing device 260 in some examples. In example embodiments, a band can vibrate at random times throughout the day, which helps a user to remember what they've learned about stress—and about their reactions to it.

FIG. 6D depicts an example of a user interacting with wearable device 202. The band or other attachment member can be formed from a material such as rubber, nylon, plastic, metal, or any other type of material suitable to receive user input. In some examples, the band can be made of a material that feels good to touch or squeeze. When a user feels tense, they may release tension by fidgeting with the band. In example embodiments, the band can be made of a material that feels good to touch or squeeze. When a user feels tense, they can release tension by fidgeting with the band.

FIG. 6E depicts an example user interaction with a remote computing device 260. The user can view physiological characteristics or responses detected by the plurality of sensors of the wearable device 202. The information can be provided on a GUI of the user's remote computing device 260 generated by the device manager. For example, the user can view his or her heart rate or electrodermal activity throughout the day on the remote computing device 260. The GUI provides a display including information to inform the user as to the use of the wristband and how it can be used to benefit the user as well. By way of example, sensor data or data derived from the sensor data such as a user's heart rate over a week or other interval can be displayed to the user can determine when their heart rate was high and think about why. In example embodiments, a band logs a user heart rate over the week. The user can look at this in a graphical user interface, to see when their heart rate was high and think about why.

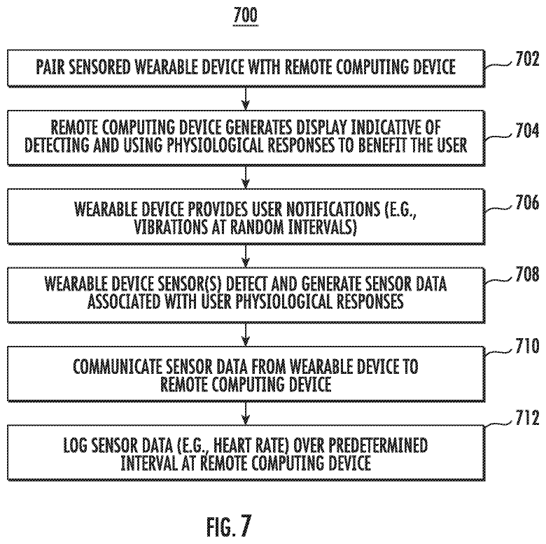

FIG. 7 is a flowchart depicting an example process 700 including communication between a wearable device and a remote computing device in accordance with example embodiments of the present disclosure. Process 700 and the other processes described herein are shown as sets of blocks that specify operations performed but are not necessarily limited to the order or combinations shown for performing the operations by the respective blocks. One or more portions of process 700, and the other processes described herein, can be implemented by one or more computing devices such as, for example, one or more computing devices 260 of a computing environment 200 as illustrated in FIG. 2 (e.g., sensing circuitry 206, processing circuitry 210, computing device 260, etc.) and/or one or more computing devices (e.g., processing circuitry 221) of wearable device 202. While in portions of the following discussion reference may be made to a particular computing environment, such reference is made for example only. The techniques are not limited to performance by one entity or multiple entities operating on one device. One or more portions of these processes can be implemented as an algorithm on the hardware components of the devices described herein.

At (702), process 700 may include pairing a wearable device with a remote computing device. For example, block 702 may include pairing a smart wristband with a plurality of sensors to a user's mobile smartphone. As an example, the pairing of a remote computing device with a wearable device can be done via a mobile application.

At (704), process 700 may include generating a graphical user interface at the remote computing device displaying an indication of detecting and using physiological responses to benefit the user. For example, the remote computing device can generate one or more graphical user interfaces including a display of beneficial information about how to manage stress or other physiological characteristics or responses associated with the user.

At (706), process 700 may include providing one or more user notifications via the wearable device. For example, the wearable device may vibrate at random intervals to remind a user of the beneficial information on how to manage stress provided by the remote computing device. According to some example aspects, the wearable device may provide visual, audio, and/or haptic responses to remind a user of the beneficial information on how to manage stress provided by the remote computing device. For example, the wearable device can provide user notifications at random intervals of time. For instance, the wearable device can notify the user at random intervals of time using a vibration pattern or a visual pattern using one or more LEDs.

At (708), process 700 includes detecting and generating sensor data. For example, the wearable device can detect and generate sensor data associated with user physiological responses. For instance, the wearable device can detect the user's heart rate and measure the user's heart rate throughout a day. In another example, the wearable device can detect the blood volume level of the user using a PPG. In another example, the wearable device can detect movement data using an IMU. In another example, the wearable device can detect fluctuations in the electrical characters of the user's skin using EDA sensors. The sensor data generated is not limited to the above examples. Any of the sensors indicated in FIGS. 3A-3C can be used to detect and generate sensor data associated with the user's physiological responses.

At (710), process 700 includes transmitting sensor data from the wearable device to a remote computing device. For example, the wearable device can communicate the detected and generated sensor data to a user's mobile smartphone. By way of example and not limitation, a wearable device may communicate sensor data to the remote computing device over a local-area-network (LAN), a wireless local-area-network (WLAN), a personal-area-network (PAN) (e.g., Bluetooth™), a wide-area-network (WAN), an intranet, the Internet, a peer-to-peer network, point-to-point network, a mesh network, and the like.

At (712), process 700 includes logging sensor data and/or other data at a remote computing device. The remote computing device may log sensor data or other data over a predetermined interval. For example, the remote computing device may log the user's heart rate over the entire day. The user can view this sensor data that has been logged at the remote computing device. In an example, the user can view the changes in the user's heart rate, EDA, or other physiological characteristics over a specific period of time.

The wearable device 202 can include a band(s) or attachment member may be made of any material that can provide visual, audio, and/or haptic input to the wearable device 202 identifying a stressful time period for the user. In an example, the user may fidget with the band of the wearable device 202 if the user feels stressed or if the physiological responses of the user are above a predetermined threshold. For example, the user may fidget with the band of the wearable device 202 if the user's PPG signals are over a predetermined threshold.

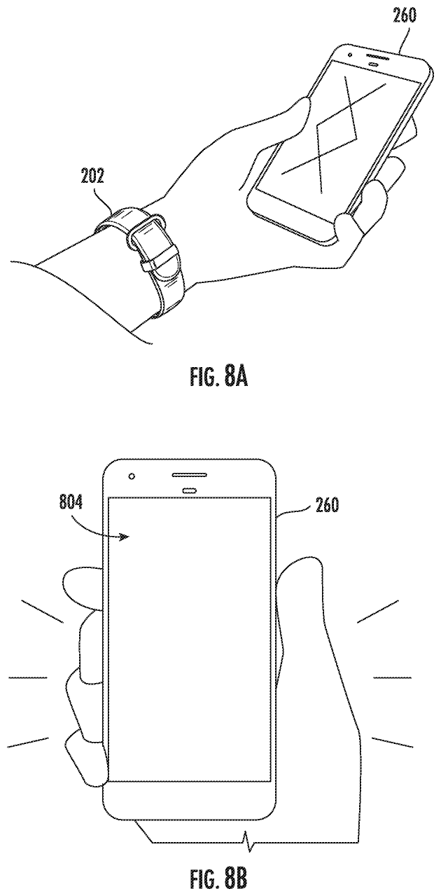

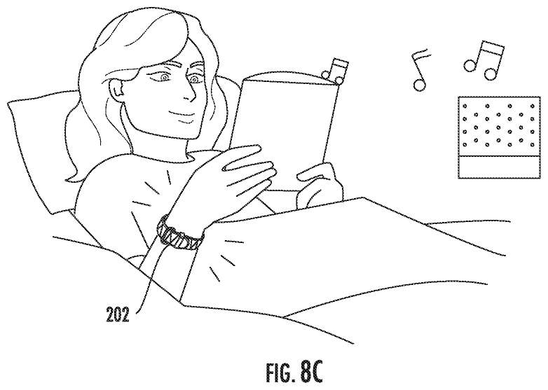

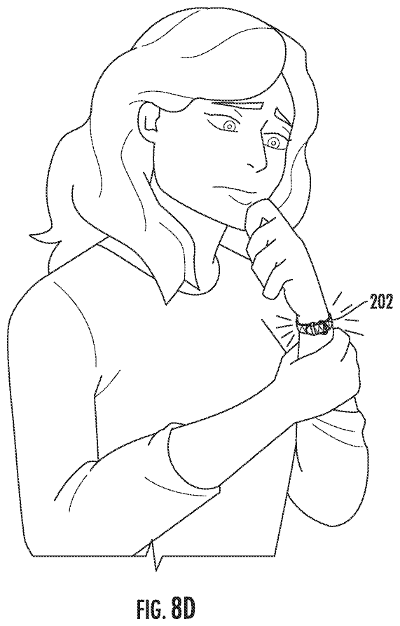

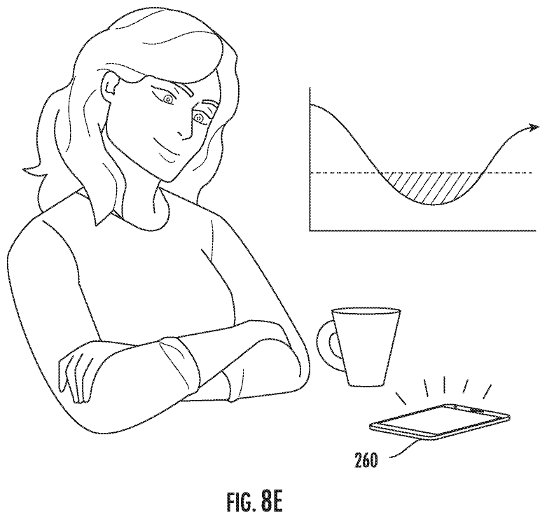

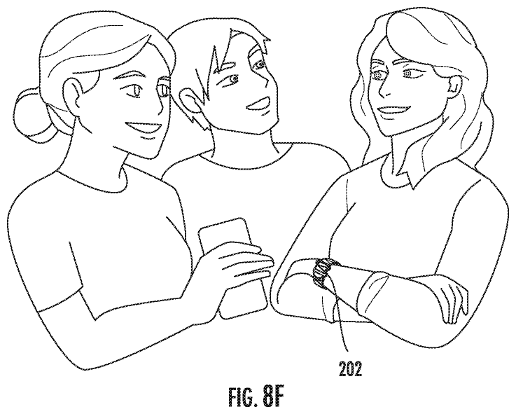

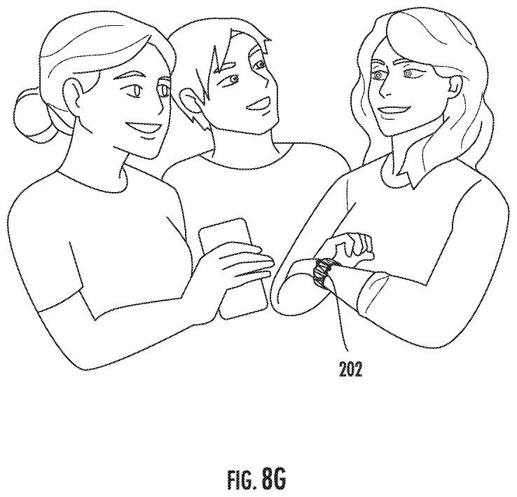

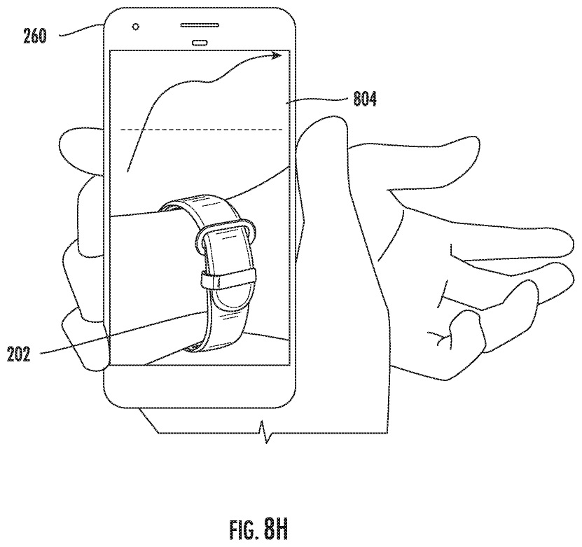

FIGS. 8A-8G depicts an example of a user interaction with a wearable device 202 and a remote computing device 260.

FIG. 8A depicts a remote computing device 260 (e.g., user's smartphone) and a wearable device 202 (e.g., smart wristband). In example embodiments, the wearable device 202 and the remote computing device 260 can be communicatively coupled using an application programming interface that enables the remote computing device 260 and the wearable device 202 to communicate. In some examples, the wearable device can include a wristband manager that can interface with the wristband to provide information to a user (e.g., through a display, audible output, haptic output, etc.) and to facilitate user interaction with the wristband. Additionally or alternatively, remote computing device 260 can include a device manager configured to generate one or more graphical user interfaces that can provide information associated with wearable device 202. By way of example, a device manager at a remote computing device can generate one or more graphical user interfaces that provide graphical depictions of sensor data or data derived from sensor data.

FIG. 8B depicts a graphical user interface (GUI) 604 displayed by the remote computing device 260 in accordance with example embodiments of the present disclosure. In this example, the GUI provides a display including information that informs the user as to the use of the wristband and how it can be used to benefit the user. By way of example, the GUI may provide an indication that the wristband can be used to detect various physiological responses associated with a user and provide information associated with the various physiological responses. The GUI may provide an indication that detecting physiological responses and providing such information may be used to benefit the user.

FIG. 8C depicts an example of a wearable device 202 generating user notifications. The user notifications can be visual, aural, and/or haptic in nature. The user notifications can be generated at periodic intervals or at random intervals. For example, the wearable device 202 may generate vibrations and/or vibration patterns at random times. The user notifications can be provided to remind to the user to think about the beneficial information pertaining to physiological responses that the was displayed by the user's remote computing device 260 in some examples.

FIG. 8D depicts an example of a user interaction with a wearable device 202 indicating a stressful time period for a user. For example, the user may fidget with the band of the wearable device 202 indicating that the user experiencing a stressful time period. In another example, the user may apply pressure on the band indicating that the user is experiencing a stressful time period. The wearable device can include one more input devices such as one or more capacitive touch sensors etc. configured to receive user input. Sensor data associated with the physiological responses of the user during the identified stressful time period can be recorded or otherwise modify the wearable device and/or remote computing device. The wearable device 202 can generate sensor data such as EDA data, heart rate data, PPG data, or other sensor data. The wristband manager or device manager can associate the sensor data with a stressful event or other physiological response. The wearable device 202 and/or the computing device can continue to record sensor data until the user provides an input indicating that the stressful time period has passed. In example embodiments, when a user is in a stressful situation, they can squeeze the band. It can record the user's body signals until the user calms down again.

FIG. 8E depicts an example of a user interaction with a remote computing device 260 to view sensor data and/or data derived from sensor data. The user can view his or her own physiological responses as detected by the plurality of sensors of the wearable device 202 on the GUI at the user's remote computing device 260. For example, the user can view his or her heart rate or electrodermal activity throughout the day on the GUI at the remote computing device 260. The GUI provides a display including information to inform the user as to the use of the wristband and how it can be used to benefit the user as well. In example embodiments, a band records body signals and episodes of stress. The user can look back at these episodes in the app, and think about the patterns. In example embodiments, over time, the band “learns” a user's body's signals during times of stress. It can “guess” when a user is stressed even before they realize it. It lets the user know by sending a signal.

FIG. 8F depicts an example of a wearable device 202 using generated sensor data to train a one or more machine-learned physiological response prediction models for a user. For example, the wearable device 202 can use generated sensor data to train a physiological response predictor. Based on a prediction of a physiological response by machine learned model, the wearable device 202 can provide a user notification of the physiological response prediction (e.g., a stressful time period).

If the user experiences a stressful time period, then the user can confirm the physiological response prediction by providing a first input (e.g., by applying pressure) to the band of the wearable device 202 as depicted in FIG. 8G. If the user does not experience a stressful time period, the user can provide a second input (e.g., by tapping or flicking the band of the wearable device 202) to indicate that the prediction was not correct. Positive and/or negative training data can be generated based on a user confirmation input to train the machine-learned physiological response predictor. In an example, the positive or negative training data is used to calculate one or more loss functions, and the loss functions in turn are used to update the one or more machine-learned physiological response predictor models. In example embodiments, a user can confirm the band is correct by squeezing it. If the band guessed wrong, a user can tap or flick it to dismiss it, and help it learn better for next time.

FIG. 8H depicts an example of generating a graphical user interface at the remote computing device 260 based on the generated sensor data and relative positions of the remote computing device 260 and wearable device 202. The relative position of the remote computing device 260 to the wearable device 202 is evaluated to determine if one or more positional constraints are satisfied. The wearable device and/or remote computing device can determine if one or more thresholds (e.g., positional constraints) are satisfied by the relative position. For instance, a positional constraint can specify a threshold distance. If the relative position indicates that the two devices are within the threshold distance, the positional constraint can be satisfied. In another example, a threshold may include a time constraint. For instance, a time constraint can specify a threshold time that the remote computing device is within a threshold distance. Other thresholds may be used such as more precise positioning of the remote computing device to the wearable device. For instance, it can be determined whether the remote computing device is positioned above (e.g., hovered over) and within a predetermined distance of the wearable device in order to determine if one or more thresholds have been satisfied. In yet another example, a positional constraint can include a relative direction of motion between the remote computing device and the wearable device. If the one or more thresholds are satisfied, the graphical user interface at the remote computing device can be updated based on the sensor data or other data generated by the wearable device and sent to the computing device. By way of example, the remote computing device can automatically generate the user interface to display sensor data or other data from the wearable device in response to determining that the one or more threshold are satisfied. In this manner, the remote computing device can provide a seamless virtual display into the insights gathered by the wearable device. In example embodiments, if a user wants to look at their body's stress reactions that day, they can hold their phone over the band to see how their stress levels have changed.

FIG. 9 is a flowchart describing an example process 900 of generating sensor data using one or more sensors of a wearable device and training a machine learned physiological response model (e.g., a detector and/or predictor) using the sensor data.

At (908), process 900 includes receiving user input user input at a wearable device identifying a stressful time period. In example embodiments, the wearable device may include one or more inputs devices configured to receive a user input indicating a time period. For example, the user can fidget with the wrist band of the smart wristwatch when the user is stressed. In another example, the user can apply pressure to the band of the smart wristwatch, the change in pressure on the band being indicative of the user's stressful time period. In some examples, the wristband can include a touch sensor such as a resistive or capacitive touch sensor configured to receive touch inputs from a user and detect gestures based on the touch input.

At (910), process 900 includes detecting one or more physiological characteristics of the user during the identified time period. At (910), process 900 can include generating sensor data indictive of the one or more physiological characteristics. For example, one or more sensors of a smart wristband may generate sensor data indicative of a user's heart rate, EDA, and/or blood pressure, among other physiological characteristics. The smart wristband can associate the sensor data with the period of stress identified by the user input to the wearable device.

At (912), process 900 includes generating training data for a machine-learned system of the wearable device. By way of example, the sensor data generated during the time period identified by the user can be automatically annotated as corresponding to stress or a stressful event. The training data can be generated locally by the wristband from sensor data generated by the wristband. The training data can be provided as an input to one or more machine-learned models of the machine-learned system at the wristband during a training period. In this manner, the generated sensor data can be provided as training data to train the machine-learned system.

At (914), process 900 includes training the machine learned system using the sensor data correlated to the time period identified by the user input at (908). One or more machine-learned models can be trained to provide one or more physiological response detection and/or prediction for the user. For example, a machine-learned detector model can be trained to detect a stressful event based on sensor data (e.g., EDA data). As another example, a machine-learned predictor model can be trained to predict a future stressful event based on sensor data.

At (918), process 900 includes communicating sensor data and/or data derived from the sensor data from the wearable device to a remote computing device. The data obtained by the remote computing device can be used to generate one or more graphical user interfaces associated with a user's physiological activity. For example, the wearable device can communicate sensor data and/or machine-learned inferences based on the sensor data to a user's mobile smartphone. By way of example and not limitation, a wearable device may communicate sensor data to the remote computing device over a local-area-network (LAN), a wireless local-area-network (WLAN), a personal-area-network (PAN) (e.g., Bluetooth™), a wide-area-network (WAN), an intranet, the Internet, a peer-to-peer network, point-to-point network, a mesh network, and the like. In an example, the sensor data communicated to the wearable device can be arranged in the form of charts and graphs to indicate the sensor data and/or changes in the sensor data.

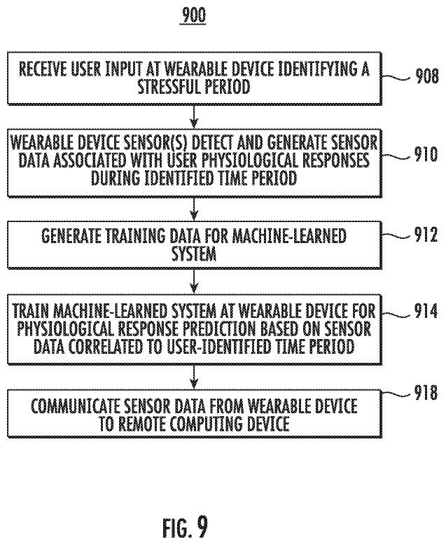

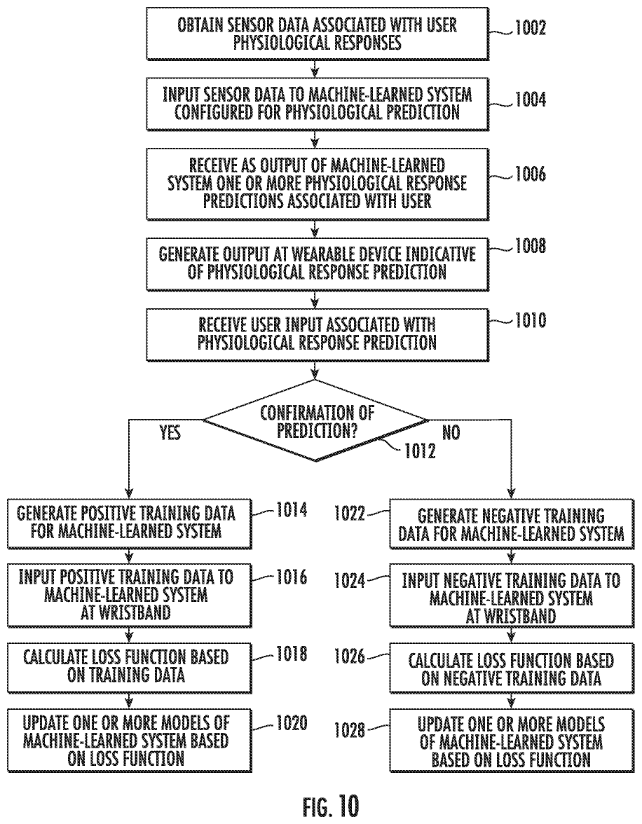

FIG. 10 is a flowchart depicting an example process 1000 of training a machine learned system including one or more machine-learned models. The machine-learned system can be trained locally at a wearable device using sensor data generated by the wearable device. In example embodiments, user confirmation of detections and/or predictions by the machine-learned system can be used to automatically annotate the sensor data to generate training data for the machine-learned system.

At (1002), process 1000 can include obtaining sensor data generated by one or more sensors of a wearable device such as a smart wristband. In example embodiments, the sensor data can be representative of one or more physiological characteristics or responses of a user. The sensor data can be generated by one or more sensors such as an EDA sensor, PPG sensor, ECG sensor, and/or an IMU. The sensor data can be indicative of the physiological responses of the user of a wearable device.

At (1004), process 1004 includes inputting sensor data into a machine learned physiological response system. The sensor data can be provided as one or more inputs to one or more machine-learned models configured for physiological response prediction. The sensor data from one or more sensors can be input into a machine-learned physiological response prediction model for instance. The sensor data from one or more sensors such as the EDA, PPG, ECG, and/or the IMU can be input into the machine-learned physiological response system.

At (1006), process 1000 includes receiving as output of the machine learned system one or more physiological response predictions associated with the user. By way of example, data indicative of a physical response prediction may be received as one or more outputs of a machine learned predictor model. Examples of physical response predictions include, but are not limited to, predictions of future stress events, predictions of future heart rate events, predictions of future sleeping events, predictions of future mood events, etc. In some examples, a prediction may indicate a future time at which the predicted response is predicted to occur.

At (1008), process 1000 includes generating an output based on the one or more physiological response predictions associated with the user. The wearable device can generate various types of outputs that are indicative of a physiological response prediction. For example, in response to a physiological event prediction, the wearable device can generate an output indicating the type of predicted physiological response and/or a time associated with the predicted physiological response. For instance, the wearable device can generate a visual, audible, and/or haptic output indicating that the user is likely to experience a stressful event in 30 minutes.

In an example, a smart wristband (e.g., device 100 in FIG. 1) may include one or more output devices configured to generate a user notification such as a visual, audible, and/or haptic response. An output device can be configured to provide a haptic response, a tactical response, an audio response, a visual response, or some combination thereof. Output devices may include visual output devices, such as one or more light-emitting diodes (LEDs), audio output devices such as one or more speakers, one or more tactile output devices, and/or one or more haptic output devices. In some examples, the one or more output devices are formed as part of the wearable device, although this is not required. In one example, an output device can include one or more devices configured to provide different types of haptic output signals. For example, the one or more haptic devices can be configured to generate specific output signals in the form of different vibrations and/or vibration patterns based on the user's stress, and/or other physical or physiological characteristics or responses. In another example, a haptic output device may tighten or loosen a wearable device with respect to a user. For example, a clamp, clasp, cuff, pleat, pleat actuator, band (e.g., contraction band), or other device may be used to adjust the fit of a wearable device on a user (e.g., tighten and/or loosen). In one example, an output device can include one or more LEDs configured to provide different types of output signals. For example, the one or more LEDs can be configured to generate patterns of light, such as by controlling the order and/or timing of individual LED activations based on the user's stress, and the user's physical and physiological responses. Other lights and techniques may be used to generate visual patterns including circular patterns. In some examples, one or more LEDs may produce different colored light to provide different types of visual indications.

At (1010), process 1000 includes receiving at the wearable device a user input associated with the physiological response prediction. For example, the user may provide a user confirmation input indicating whether the physiological response prediction was accurate. In some examples, the user may provide a first input to positively confirm a physiological response prediction and a second input to negatively confirm a physiological response protection provided by the machine-learned physiological response predictor. For example, the user can provide one or more inputs to indicate whether the user experienced stress in accordance with a stress prediction provided by the wearable device. By way of example, a user may provide a tapor flickinput to the band of a smart wristband as a user confirmation signal.

At (1012), process 1000 includes determining whether the physiological response prediction was confirmed by the user.

If the physiological response prediction is confirmed, process 1000 continues at (1014), where process 1000 includes generating positive training data for the machine-learned physiological response prediction system. In example embodiments, positive training data can be generated by annotating or otherwise associating the sensor data with the predicted physiological response. For example, the training data can include sensor data and annotation data indicating that the sensor data corresponds to one of our stressful events.

At (1016), process 1000 includes providing the positive training data as input to the machine-learned physiological response prediction system at the wearable device. In some examples, the sensor data and annotation data can be provided as an input to the machine learned physiological response prediction system during training. For example, if positive input is received from the user, positive training data is generated, and the positive training data is further used to train the machine-learned physiological response prediction system.

At (1018), one or more loss function parameters can be determined for the machine-learned physiological response prediction system based on the positive training data. In some examples, one or more loss function parameters can be calculated using a loss function based on an output of one or more machine learned models. For example, annotation data can provide a ground truth that is utilized by a training system to calculate the one or more loss function parameters in response to a prediction from the model based on the corresponding sensor data.

At (1020), process 1000 may include updating one or more models of the machine-learned system based on the calculated loss function. By way of example, one or more weights or other attributes of a machine learned model may be modified in response to the loss function.

Returning to (1012), if the command physiological response prediction is not confirmed process 1000 continues at (1022), where negative training data is generated for the machine-learned physiological response prediction system. In example embodiments, negative training data can be generated by annotating or otherwise indicating that the sensor data does not correspond to desired physiological response for the system to detect. For example, the training data can include sensor data and annotation data indicating that the sensor data does not correspond to one of our stressful events.

At (1024), process 1000 includes providing the negative training data as input to the machine-learned physiological response prediction system at the wearable device. In some examples, the sensor data and annotation data can be provided as an input to the machine learned physiological response prediction system during training. For example, if negative input is received from the user, negative training data can be generated, and the negative training data used to train the machine-learned physiological response prediction system.

At (1026), one or more loss function parameters can be determined for the machine-learned physiological response prediction system based on the negative training data. In some examples, one or more loss function parameters can be calculated using a loss function based on an output of one or more machine learned models. For example, annotation data can provide a ground truth that is utilized by a training system to calculate the one or more loss function parameters in response to a prediction from the model based on the corresponding sensor data.

At (1028), one or more models of the machine-learned system can be updated based on the calculated loss function. By way of example, one or more weights or other attributes of a machine learned model may be modified in response to the loss function.

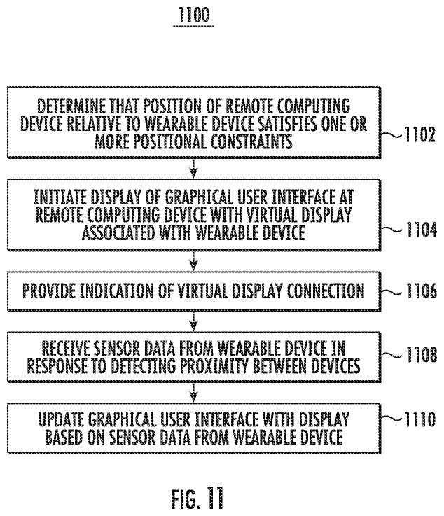

FIG. 11 is a flowchart for an example process 1100 of generating and displaying a graphical user interface based on sensor data from a wearable device.

At (1102), process 1000 includes detecting a proximity event associated with a wearable device and a remote computing device. In some examples, a proximity event can be detected using one or more proximity constraints. Proximity constraints can include, but are not limited to positional constraints and time constraints. In some examples, process 1000 includes determining that a position of the remote computing device relative to the wearable device satisfies one or more positional constraints and/or one or more time constraints. In one example, a positional constraint can be applied to determine whether the wearable device and remote computing device are within a predetermined proximity of each other. In another example, a positional constraint can be applied to determine whether the remote computing device is hovered a predetermined distance over the wearable device. In yet another example, a positional constraint can be applied to determine a relative direction of motion between the remote computing device and the wearable device. A time constraint can be applied to determine whether the remote computing device and wearable device are within a threshold distance for a threshold time. In some examples, the wearable device can determine whether the proximity constraint(s) is satisfied. In other examples, the remote computing device can determine whether the proximity constraint(s) is satisfied.

At (1104), process 1000 includes initiating a display of a graphical user interface at a remote computing device in response to the wearable device satisfying the one or more proximity constraints. The graphical user interface may be displayed automatically in response to a determination that the proximity constraints are satisfied. The remote computing device can initiate the display of the graphical user interface in response to detecting a proximity event in some examples. The remote computing device can initiate and/or update the display of the graphical user interface in response to receiving data associated with one or more physiological characteristics of a user as may be determined from one or more sensors of the wearable device. In some examples, the wearable device may initiate the display of the graphical user interface by transmitting data indicative of the proximity event and/or the data associated with the one or more physiological characteristics of the user.

At (1106), process 1000 includes providing an indication of a virtual display connection between the wearable device and the remote computing device. A virtual display connection can be established between the remote computing device and/or the wearable device. The virtual display connection can be established by the remote computing device and/or the wearable device. The connection can be established in response to detecting the proximity event in some examples. Additionally and/or alternatively, the connection can be established in response to the transmission and/or receipt of data associated with the physiological characteristics of the user. According to some aspects, a graphical user interface may be displayed at the remote computing device to virtually provide a display in association with the wearable device. By way of example, the graphical user interface may provide a first display indicating a virtual display connection between the remote computing device and the wearable device. In one example, the virtual display may provide a first display including a real-time depiction of the position of the body part on which the wearable device is worn and the wearable device. For example, the graphical user interface of the remote computing device may display imagery (e.g., one or more images or videos) captured by one or more image sensors (e.g, cameras) depicting the real-time position of the user's hand and of the smart wristband on the hand of the user.

In various examples, the remote computing device can provide an indication of a virtual display connection between the wearable device and the remote computing device and/or the wearable device can provide an indication of a virtual display connection between the wearable device and the remote computing device. An indication of a virtual display connection can be provided by the graphical user interface of the remote computing device and/or one or more output devices of the wearable device. For example, a smart wristband may provide an indication of a virtual display connection or a user smartphone may provide an indication of a virtual display connection on the one or more output devices of the smart wristband.

At (1108), data associated with the one or more physiological characteristics is received from the wearable device. In some examples, sensor data is received from the wearable device in response to determining that the position of the remote computing device relative to the wristband satisfies the one or more proximity constraints. The sensor data can be automatically communicated by the wearable device to the remote computing device in response to determining that the relative position satisfies the proximity constraints. In other examples, data derived from the sensor data can be transmitted from the wearable device to the remote computing device.

At (1110), the graphical user interface at the remote computing device is updated with a display based on the sensor data and/or other data received from the wearable device. For example, if a user hovers the remote computing device (e.g., smartphone) over a smart wristband satisfying the one or more positional constraints, the graphical user interface of the remote computing device may present a virtual display showing the real-time position of the user's hand and of the smart wristband on the hand of the user.

In one example, the wearable device may update the graphical user interface at the remote computing device to enable a virtual display associated with the wearable device based on the sensor data from the wearable device. The virtual display may depict sensor data and/or data derived from the sensor data.

The remote computing device can update the virtual display based on the sensor data from the wearable device.

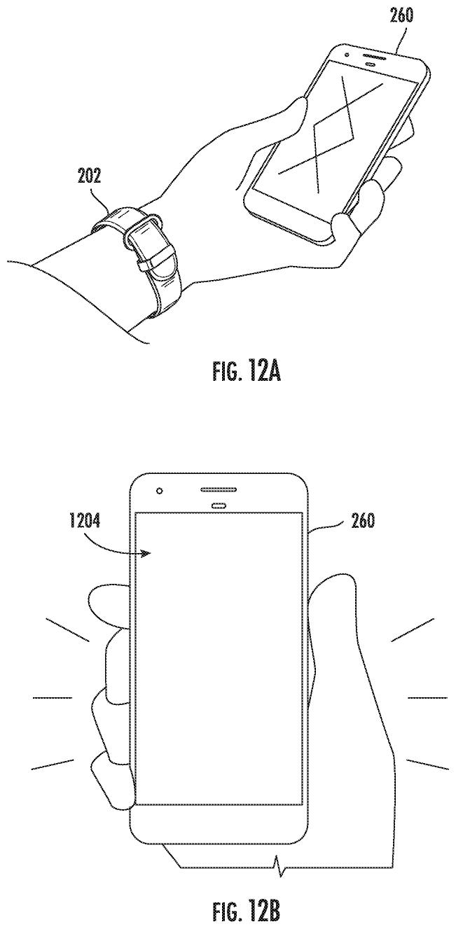

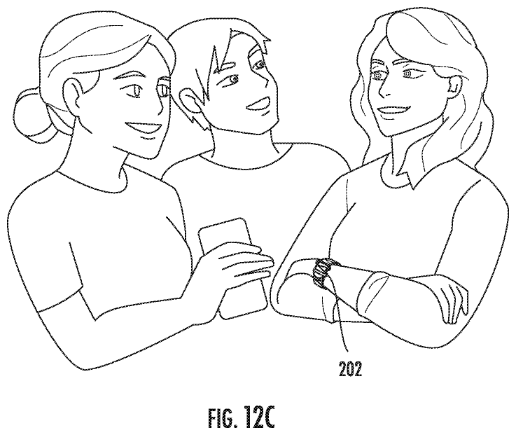

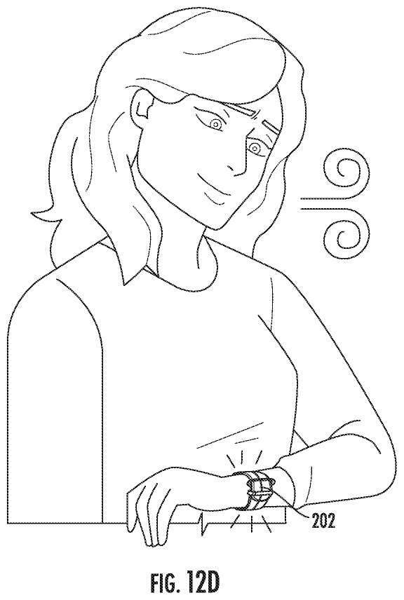

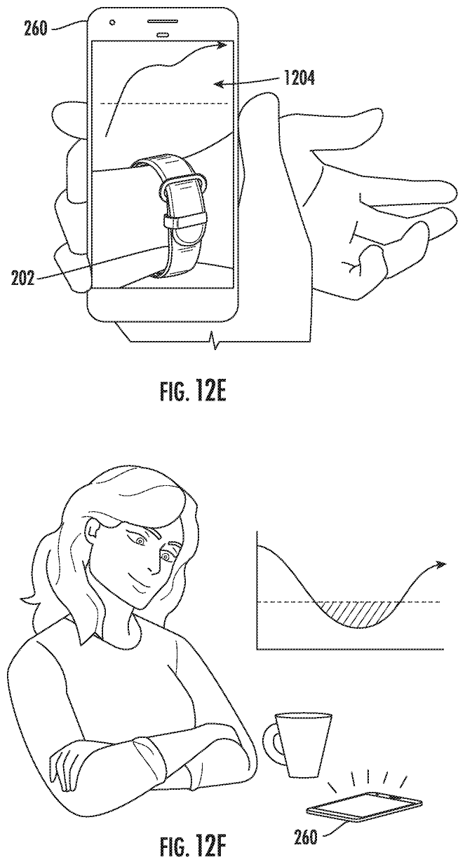

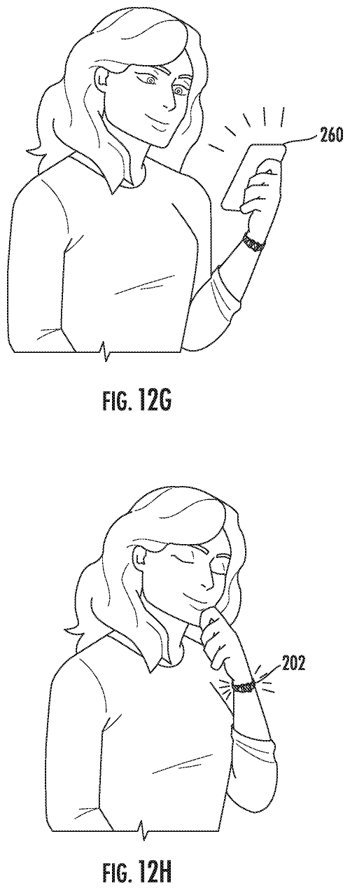

FIG. 12A-12H depict an example user interaction with a wearable device 202 and a remote computing device 260.

FIG. 12A, depicts an example of communicatively coupling between a remote computing device 260 (e.g., user's smartphone) and a wearable device 202 (e.g., smart wristband) to set up the wearable device 202. For example, the wearable device 202 and the remote computing device 260 can be communicatively coupled using an application programming interface that enables the remote computing device 260 and the wearable device 202 to communicate.

FIG. 12B depicts a graphical user interface (GUI) displayed by the remote computing device 260 in accordance with example embodiments of the present disclosure. In this example, the GUI provides a display including information that informs the user as to the use of the wristband and how it can be used to benefit the user. By way of example, the GUI may provide an indication that the wristband can be used to detect various physiological responses associated with a user and provide information associated with the various physiological responses. The GUI may provide an indication that detecting physiological responses and providing such information may be used to benefit the user.

FIG. 12C depicts an example scenario in which a machine learned physiological predictor predicts a user's future stress event based on the sensor data from the one or more sensors of the wearable device 202. In example embodiments, the wearable device can sense when a user is starting to become stressed, even before the user is aware of it. The wearable device can alert the user by sending a gentle signal.

FIG. 12D depicts an example user interaction with a wearable device to generate soothing signals to calm the user at the end of a user stress event. The wearable device can determine if a user is starting to calm down after stress. It can send a soothing signal to help the user recover quickly. Wearable device 202 can generate one or more soothing signals using one or more output devices, based on a one or more machine learned model's detection of a user calming event and/or end of user stress event. For example, after the user has a stress event, and the wearable device 202 detects a user calming event, the wearable device 202 can output soothing signals to sooth the user. If the wearable device 202 is a smart wristband, the smart wristband can generate soothing signals (e.g., a smooth vibration along the band) to sooth the user. In an example, if the wearable device 202 is a smart wristband (e.g., device 100 in FIG. 1), the output can be generated on the wristband of the wearable smart wristband (e.g., attachment member 150 in FIG. 1A). In an example, the soothing signal may comprise a smooth vibration along the band of the smart wristband. In another example the soothing signal may comprise a soothing audio signal. In another example, the soothing signal may comprise a soothing visual signal.

FIG. 12E depicts an example of generating a graphical user interface at the remote computing device 260 based on the generated sensor data and relative positions of the remote computing device 260 and wearable device 202. If the relative position of the remote computing device 260 to the wearable device 202 satisfies one or more positional constraints, the wearable device 202 may establish a virtual display connection with the remote computing device 260, and update a graphical user interface at the remote computing device 260 to enable a virtual display associated with the wearable device 202. In an example, the relative position of the remote computing device 260 to the wearable device 202 satisfies the one or more positional constraints, then the remote computing device 260 may establish a virtual display connection with the wearable device 202, and update the graphical user interface at the remote computing device 260 to enable a virtual display associated with the wearable device 202. For example, if a user wants to look at their body's stress reactions that day, they can hold their phone over the band to see how their stress levels have changed.

FIG. 12F depicts an example of a user interaction with a remote computing device 260 to view sensor data and/or data derived from sensor data. The user can view his or her own physiological responses as detected by the plurality of sensors of the wearable device 202 on the GUI at the user's remote computing device 260. For example, the user can view his or her heart rate or electrodermal activity throughout the day on the GUI at the remote computing device 260. The GUI provides a display including information to inform the user as to the use of the wristband and how it can be used to benefit the user as well. The band can record a user's body signals and episodes of stress over time. The user can look back at these episodes using the remote computing device, and think about the patterns.

FIG. 12G depicts an example of generation of data indicative of a pattern of stress associated with a user. The data indicative of pattern of stress associated with user can be generated by one or more machine-learned models based at least in part on sensor data. In an example, the data indicative of pattern of stress associated with user can be displayed on the remote computing device 260 in the form of charts and graphs to indicate the pattern of stress associated with the user. For example, the pattern of stress associated with a user may be displayed to the user based on the time of day leading up to the one or more stress events. In another example, the pattern of stress associated with a user may be displayed to the user indicative of the physiological response changes during one or more stress events. The band can use artificial intelligence to identify situations in which a user becomes stressed. The remote computing device (e.g. device manager) can generate data to teach a user about these patterns and offer the user resources for coping.

FIG. 12H depicts an example of a user interaction with the wearable device 202 generating soothing signals for the user on output devices at the user's request. The wearable device 202 receives user input indicative of a user request or indicative of a user stress event. An input device such as a touch input device can be utilized to enable user to provide input to the wearable device 202. An input device such as a touch device can be utilized to enable user to view the output or cause a response by the wearable device 202. The wearable device 202 can determine one or more soothing output responses and generate one or more signals to cause one or more output devices to generate the output responses to sooth the user. In an example, if the wearable device 202 is a smart wristband (e.g., device 100 in FIG. 1), the output in response to the user input indicative of user stress event or a user input that is a user request for soothing signals can be generated on the wristband of the wearable smart wristband (e.g., attachment member 150 in FIG. 1A). In an example, the soothing signal may comprise a smooth vibration along the band of the smart wristband. In another example the soothing signal may comprise a soothing audio signal. In another example, the soothing signal may comprise a soothing visual signal.

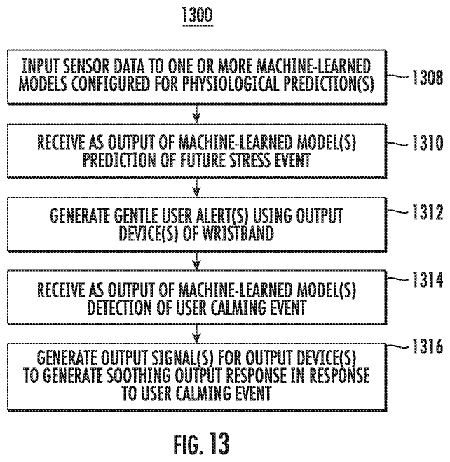

FIG. 13 is a flowchart depicting an example process 1300 of using one or more machine-learned physiological response prediction models to predict user physiological responses based on sensor data.

At (1308), process 1300 may include providing sensor data as input to one or more machine-learned models configured for physiological response predictions.

At (1310), process 1300 may include receiving as output of the one or more machine learned physiological response prediction models, data indicative of a prediction of a future stress event in association with a user. For example, based on the sensor data input into the one or more machine learned physiological response prediction models, the model(s) may predict that the user is likely to experience a future stress event at a particular time in the future.

At (1312), process 1300 may include generating one or more gentle user alerts using one or more output devices of the wearable device. The one or more user alerts can be generated automatically in response prediction of a future stress event as output of the monomer machine learned models, based on the one or more machine learned physiological response predictions of a future stress event for a user. For example, if the one or more machine learned physiological response prediction models predicts that the user will experience a future stress event, the smart wristband can generate gentle user alerts (e.g., a smooth vibration along the band) indicative of the future stress event for the user. In an example, if the wearable device is a smart wristband (e.g., device 100 in FIG. 1), the output can be generated on the wristband of the wearable smart wristband (e.g., attachment member 150 in FIG. 1A). In an example, the gentle user alert may comprise a smooth vibration along the band of the smart wristband. In another example the gentle user alert may comprise a soothing audio signal. In another example, the gentle user alert may comprise a soothing visual signal. The band or other attachment member can be formed from a material such as rubber, nylon, plastic, metal, or any other type of material suitable to send and receive visual, audible, and/or haptic responses. An output device can generate the output indicative of user's physiological response prediction. An output device can be configured to provide a haptic response, a tactical response, an audio response, a visual response, or some combination thereof. Output devices may include visual output devices, such as one or more light-emitting diodes (LEDs), audio output devices such as one or more speakers, one or more tactile output devices, and/or one or more haptic output devices. In some examples, the one or more output devices are formed as part of the wearable device, although this is not required. In one example, an output device can include one or more devices configured to provide different types of haptic output signals. For example, the one or more haptic devices can be configured to generate specific output signals in the form of different vibrations and/or vibration patterns based on the user's stress, and the user's physical and physiological responses. In another example, output devices may include a haptic output device such as may tighten or loosen a wearable device with respect to a user. For example, a clamp, clasp, cuff, pleat, pleat actuator, band (e.g., contraction band), or other device may be used to adjust the fit of a wearable device on a user (e.g., tighten and/or loosen). In one example, an output device can include one or more LEDs configured to provide different types of output signals. For example, the one or more LEDs can be configured to generate patterns of light, such as by controlling the order and/or timing of individual LED activations based on the user's stress, and the user's physical and physiological responses. Other lights and techniques may be used to generate visual patterns including circular patterns. In some examples, one or more LEDs may produce different colored light to provide different types of visual indications.

At (1314), process 1300 may include receiving as an output of one or more machine learned models a detection of a user calming event.

At (1316), process 1300 may include generating one or more soothing signals using one or more output devices of the wearable device in response to the detection of the user calming event. For example, after the user has a stress event, and the wearable device detects a user calming event, the wearable device can output soothing signals to sooth the user. If the wearable device is a smart wristband, the smart wristband can generate soothing signals (e.g., a smooth vibration along the band) to sooth the user. In an example, if the wearable device is a smart wristband (e.g., device 100 in FIG. 1), the output can be generated on the wristband of the wearable smart wristband (e.g., attachment member 150 in FIG. 1A). In an example, the soothing signal may comprise a smooth vibration along the band of the smart wristband. In another example the soothing signal may comprise a soothing audio signal. In another example, the soothing signal may comprise a soothing visual signal. The wristband or other attachment member can be formed from a material such as rubber, nylon, plastic, metal, or any other type of material suitable to send and receive visual, audible, and/or haptic responses. An output device can generate the output indicative of user's physiological response prediction. An output device can be configured to provide a haptic response, a tactical response, an audio response, a visual response, or some combination thereof. Output devices may include visual output devices, such as one or more light-emitting diodes (LEDs), audio output devices such as one or more speakers, one or more tactile output devices, and/or one or more haptic output devices. In some examples, the one or more output devices are formed as part of the wearable device, although this is not required. In one example, an output device can include one or more devices configured to provide different types of haptic output signals. For example, the one or more haptic devices can be configured to generate specific output signals in the form of different vibrations and/or vibration patterns based on the user's stress, and the user's physical and physiological responses. In another example, output devices may include a haptic output device such as may tighten or loosen a wearable device with respect to a user. For example, a clamp, clasp, cuff, pleat, pleat actuator, band (e.g., contraction band), or other device may be used to adjust the fit of a wearable device on a user (e.g., tighten and/or loosen). In one example, an output device can include one or more LEDs configured to provide different types of output signals. For example, the one or more LEDs can be configured to generate patterns of light, such as by controlling the order and/or timing of individual LED activations based on the user's stress, and the user's physical and physiological responses. Other lights and techniques may be used to generate visual patterns including circular patterns. In some examples, one or more LEDs may produce different colored light to provide different types of visual indications.

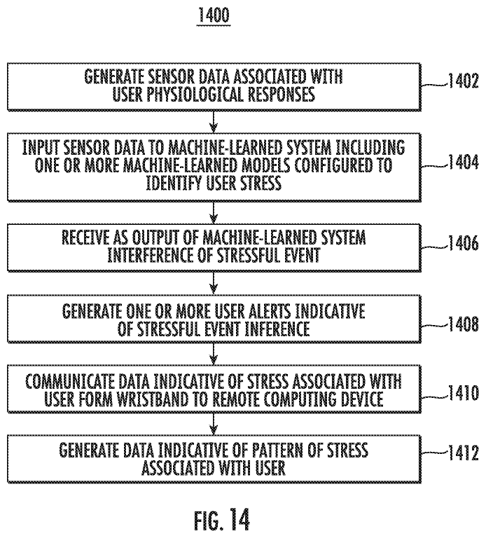

FIG. 14 is a flowchart depicting an example process 1400 of generating data indicative of a pattern of stress associated with a user in accordance with example embodiments of the present disclosure.

At (1402), sensor data associated with one or more physiological responses or other characteristics of a user are generated based on the output of one or more sensors but wearable device. For example, a sensor on a smart wristband comprising one or more sensors can detect and generate sensor data indicative of a user's heart rate, EDA, and/or blood pressure, among other physiological responses.

At (1404), sensor data is input into the one or more machine-learned systems configured to identify user stress. In an example, the sensor data is input in a physiological response system configured to attack and/or predict user stress events based at least in part on the sensor data.

At (1406), data indicative of one or more inferences associated with stressful events is received as output from the one or more machine learned models. For example, an inference of stressful events received from the one or more machine learned systems can comprise an indication of a future stressful event. In another example, an inference of stressful events received from the one or more machine learned systems can comprise the detection of a stressful event being experienced by the user. In another example, an inference of stressful events received from the one or more machine learned systems can comprise an indication that the user stress event has ended. In another example, an inference of stressful events received from the one or more machine learned systems can comprise a detection of a user calming event. In another example, an inference of stressful events received from the one or more machine learned systems can comprise a prediction of a user calming event.

At (1408), one or more user alerts indicative of an inference of a stress event are generated.

At (1410), data indicative of stress associated with the user is communicated to the remote computing device 260 from the wearable device 202 (e.g., smartphones).

At (1412), data indicative of a pattern of stress associated with a user is generated based at least in part on sensor data and/or output data from one or more of the machine-learned models. The data indicative of pattern of stress associated with user can be generated by the remote computing device and/or the wearable device.

In an example, the data indicative of pattern of stress associated with user can be displayed on the remote computing device in the form of charts, graphs, and/or other representations to indicate the pattern of stress associated with the user. For example, the pattern of stress associated with a user may be displayed to the user based on the time of day leading up to the one or more stress events. In another example, the pattern of stress associated with a user may be displayed to the user indicative of the physiological response changes during one or more stress events.

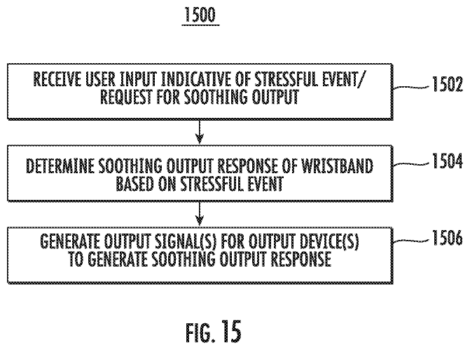

FIG. 15 is a flowchart depicting an example process 1500 of generating output signals using output devices at the request of a user.

At (1502), process 1500 includes receiving user input indicative of a stressful event and/or a request for one or more outputs by the wearable device. In an example, the user input may be indicative of a stressful user event. In another example, the user input may be a request for soothing signals by the user. An input device such as a touch input device can be utilized to enable a user to provide input to the wearable device. An input device such as a touch input device can be utilized to enable a user to view the output from the wearable device.

At (1504), one or more soothing output responses are determined based on the stressful event or other user input provided at (1502). By way of example, a device manager at the wristband may determine an appropriate output response associated with the identified stressful event.

At (1506), the device manager generates one or more output signals for one or more output devices of the wristband. The one more output signals can cause the one or more output devices to generate the determined soothing output response. The wearable device can generate the appropriate soothing output response in response to the output signals. By way of example, a smart wristband can generate soothing signals (e.g., a smooth vibration along the band) to sooth the user. In an example, if the wearable device is a smart wristband (e.g., device 100 in FIG. 1), the output can be generated on the wristband of the wearable smart wristband (e.g., attachment member 150). In an example, the soothing signal may comprise a smooth vibration along the band of the smart wristband. In another example the soothing signal may comprise a soothing audio signal. In another example, the soothing signal may comprise a soothing visual signal. The wristband or other attachment member can be formed from a material such as rubber, nylon, plastic, metal, or any other type of material suitable to send and receive visual, audible, and/or haptic responses. An output device can be configured to provide a haptic response, a tactical response, an audio response, a visual response, or some combination thereof. Output devices may include visual output devices, such as one or more light-emitting diodes (LEDs), audio output devices such as one or more speakers, one or more tactile output devices, and/or one or more haptic output devices. In some examples, the one or more output devices are formed as part of the wearable device, although this is not required. In one example, an output device can include one or more devices configured to provide different types of haptic output signals. For example, the one or more haptic devices can be configured to generate specific output signals in the form of different vibrations and/or vibration patterns based on the user's stress, and the user's physical and physiological responses. In another example, output devices may include a haptic output device such as may tighten or loosen a wearable device with respect to a user. For example, a clamp, clasp, cuff, pleat, pleat actuator, band (e.g., contraction band), or other device may be used to adjust the fit of a wearable device on a user (e.g., tighten and/or loosen). In one example, an output device can include one or more LEDs configured to provide different types of output signals. For example, the one or more LEDs can be configured to generate patterns of light, such as by controlling the order and/or timing of individual LED activations based on the user's stress, and the user's physical and physiological responses. Other lights and techniques may be used to generate visual patterns including circular patterns. In some examples, one or more LEDs may produce different colored light to provide different types of visual indications.

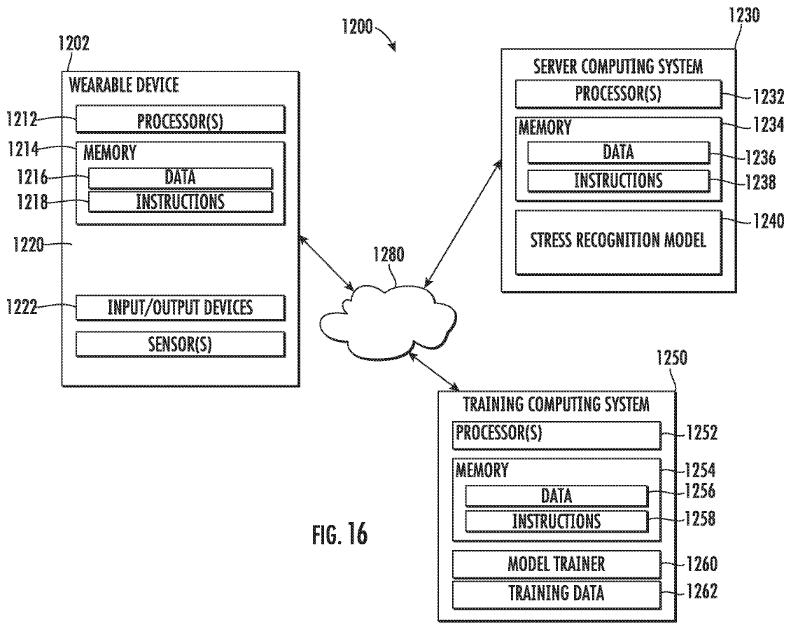

FIG. 16 depicts a block diagram of an example computing system 1200 that can perform inference generation according to example embodiments of the present disclosure. The system 1200 includes a wearable device 1202, a server computing system 1230, and a training computing system 1250 that are communicatively coupled over a network 1280.

The wearable device 1202 can be any type of a wearable device, such as, for example, a smart wristband, an ankleband, a headband, among others.

The wearable device 1202 includes one or more processors 1212 and a memory 1214. The one or more processors 1212 can be any suitable processing device (e.g., a processor core, a microprocessor, an ASIC, a FPGA, a controller, a microcontroller, etc.) and can be one processor or a plurality of processors that are operatively connected. The memory 1214 can include one or more non-transitory computer-readable storage mediums, such as RAM, ROM, EEPROM, EPROM, flash memory devices, magnetic disks, etc., and combinations thereof. The memory 1214 can store data 1216 and instructions 1218 which are executed by the processor 1212 to cause the wearable device 1202 to perform operations.

The wearable device can also include one or more sensors connected by sensor circuitry. The wearable device 1202 can also include one or more user input devices 1222 that receive user input. For example, the user input devices 1222 can be a touch-sensitive component (e.g., a capacitive touch sensor) that is sensitive to the touch of a user input object (e.g., a finger or a stylus). The touch-sensitive component can serve to implement a virtual keyboard. Other example user input components include a microphone, a traditional keyboard, or other means by which a user can provide user input.

The server computing system 1230 includes one or more processors 1232 and a memory 1234. The one or more processors 1232 can be any suitable processing device (e.g., a processor core, a microprocessor, an ASIC, a FPGA, a controller, a microcontroller, etc.) and can be one processor or a plurality of processors that are operatively connected. The memory 1234 can include one or more non-transitory computer-readable storage mediums, such as RAM, ROM, EEPROM, EPROM, flash memory devices, magnetic disks, etc., and combinations thereof. The memory 1234 can store data 1236 and instructions 1238 which are executed by the processor 1232 to cause the server computing system 1230 to perform operations.

In some implementations, the server computing system 1230 includes or is otherwise implemented by one or more server computing devices. In instances in which the server computing system 1230 includes plural server computing devices, such server computing devices can operate according to sequential computing architectures, parallel computing architectures, or some combination thereof.

The training computing system 1250 can include a model trainer 1260 that trains one or more models configured for physiological response detections and/or physiological response predictions stored at the wearable device 1202 and/or the server computing system 1230 using various training or learning techniques, such as, for example, backwards propagation of errors. In other examples as described herein, training computing system 1250 can train one or more machine learned models prior to deployment for sensor detection at the wearable device 1202 or server computing system 1230. The one or more machine-learned models can be stored at training computing system 1250 for training and then deployed to wearable device 1202 and server computing system 1230. In some implementations, performing backwards propagation of errors can include performing truncated backpropagation through time. The model trainer 1260 can perform a number of generalization techniques (e.g., weight decays, dropouts, etc.) to improve the generalization capability of the models being trained.

The model trainer 1260 includes computer logic utilized to provide desired functionality. The model trainer 1260 can be implemented in hardware, firmware, and/or software controlling a general purpose processor. For example, in some implementations, the model trainer 1260 includes program files stored on a storage device, loaded into a memory and executed by one or more processors. In other implementations, the model trainer 1260 includes one or more sets of computer-executable instructions that are stored in a tangible computer-readable storage medium such as RAM hard disk or optical or magnetic media.

The network 1280 can be any type of communications network, such as a local area network (e.g., intranet), wide area network (e.g., Internet), or some combination thereof and can include any number of wired or wireless links. In general, communication over the network 1280 can be carried via any type of wired and/or wireless connection, using a wide variety of communication protocols (e.g., TCP/IP, HTTP, SMTP, FTP), encodings or formats (e.g., HTML, XML), and/or protection schemes (e.g., VPN, secure HTTP, SSL).

FIG. 16 illustrates one example computing system that can be used to implement the present disclosure. Other computing systems can be used as well. For example, in some implementations, the wearable device 1202 can include the model trainer 1260 and the training data 1262. In such implementations, the one or more machine learned models can be both trained and used locally at the wearable device 1202. In some of such implementations, the wearable device 1202 can implement the model trainer 1260 to personalize the model heads 1220 based on user-specific data.

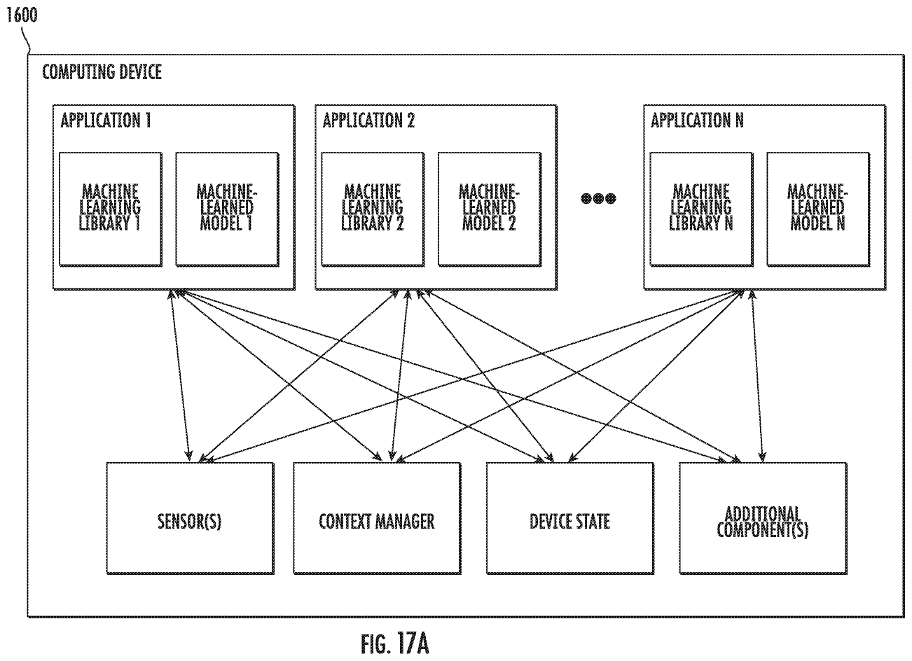

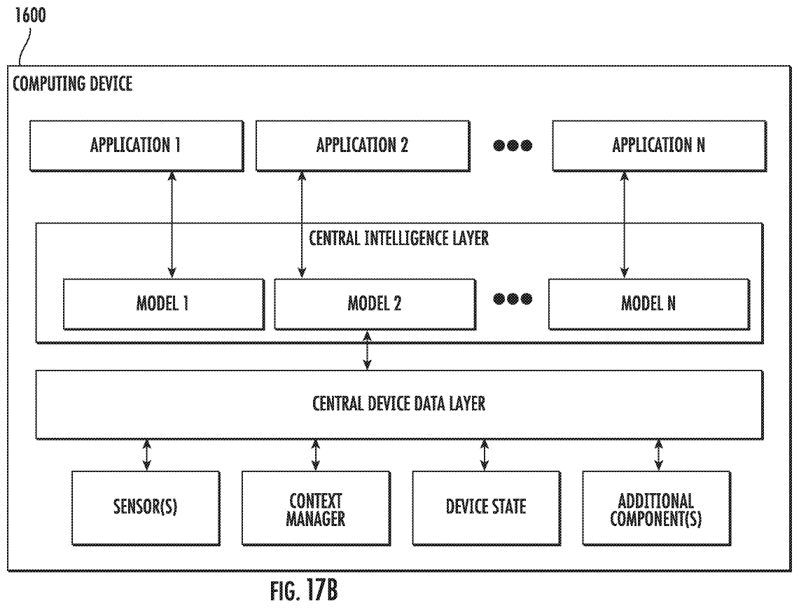

FIG. 17A depicts a block diagram of an example computing device 1600 that performs according to example embodiments of the present disclosure. The computing device 1600 can be a wearable device or a server computing device.

The computing device 1600 includes a number of applications (e.g., applications 1 through N). Each application contains its own machine learning library and machine-learned model(s). For example, each application can include a machine-learned model. Example applications include a text messaging application, an email application, a dictation application, a virtual keyboard application, a browser application, etc.

As illustrated in FIG. 17A, each application can communicate with a number of other components of the computing device, such as, for example, one or more sensors, a context manager, a device state component, and/or additional components. In some implementations, each application can communicate with each device component using an API (e.g., a public API). In some implementations, the API used by each application is specific to that application.

FIG. 17B depicts a block diagram of an example computing device 1700 that performs according to example embodiments of the present disclosure. The computing device 1600 can be a wearable device or a server computing device.

The computing device 1700 includes a number of applications (e.g., applications 1 through N). Each application is in communication with a central intelligence layer. Example applications include a text messaging application, an email application, a dictation application, a virtual keyboard application, a browser application, etc. In some implementations, each application can communicate with the central intelligence layer (and model(s) stored therein) using an API (e.g., a common API across all applications).

The central intelligence layer includes a number of machine-learned models. For example, as illustrated in FIG. 17B, a respective machine-learned model (e.g., a model) can be provided for each application and managed by the central intelligence layer. In other implementations, two or more applications can share a single machine-learned model. For example, in some implementations, the central intelligence layer can provide a single model (e.g., a single model) for all of the applications. In some implementations, the central intelligence layer is included within or otherwise implemented by an operating system of the computing device 1800.

The central intelligence layer can communicate with a central device data layer. The central device data layer can be a centralized repository of data for the computing device 1600. As illustrated in FIG. 17B, the central device data layer can communicate with a number of other components of the computing device, such as, for example, one or more sensors, a context manager, a device state component, and/or additional components. In some implementations, the central device data layer can communicate with each device component using an API (e.g., a private API).

FIG. 18 depicts a block diagram of a computing device 1600 including an example machine-learned system according to example embodiments of the present disclosure. In some implementations, the machine learned system includes a machine learned physiological response predictor that is is trained to receive a set of input data 1604 descriptive of a sensor data indicative of user's physiological responses generated from one or more sensors 204, and, as a result of receipt of the input data 1604, provide output data 1606 that is indicative of one or more predicted physiological responses such as a user stress event, sleep event, mood event, etc.

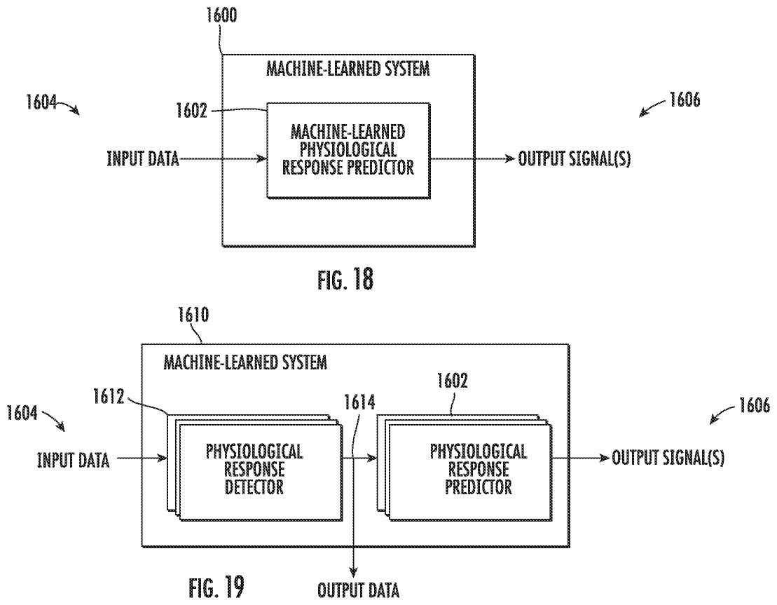

FIG. 19 depicts a block diagram of a computing device 1600 including an example machine-learned system according to example embodiments of the present disclosure. In some implementations, the machine learned system includes a machine learned physiological response detector and a machine learned physiological response predictor. The machine learned models can be trained to receive a set of input data 1604 descriptive of a sensor data indicative of user's physiological responses generated from one or more sensors 204, and, as a result of receipt of the input data 1604, provide output data 1606 that is indicative of one or more detected and/or predicted physiological responses such as a user stress event, sleep event, mood event, etc.

The technology discussed herein makes reference to servers, databases, software applications, and other computer-based systems, as well as actions taken and information sent to and from such systems. One of ordinary skill in the art will recognize that the inherent flexibility of computer-based systems allows for a great variety of possible configurations, combinations, and divisions of tasks and functionality between and among components. For instance, server processes discussed herein may be implemented using a single server or multiple servers working in combination. Databases and applications may be implemented on a single system or distributed across multiple systems. Distributed components may operate sequentially or in parallel.

While the present subject matter has been described in detail with respect to specific example embodiments thereof, it will be appreciated that those skilled in the art, upon attaining an understanding of the foregoing may readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art.

What is claimed is:

1. A wearable device comprising:
one or more sensors configured to generate data associated with one or more physiological characteristics of a user of the wearable device; and
one or more control circuits configured to obtain the data associated with the one or more physiological characteristics of the user and transmit the data to a remote computing device in response to detecting a proximity event associated with the wearable device and the remote computing device.

2. The wearable device of claim 1, wherein:
the remote computing device is configured to generate a graphical user interface including a representation of the data in response to detecting the proximity event.

3. The wearable device of claim 2, wherein the one or more control circuits are configured to, in response to detecting the proximity event:
establish a virtual display connection with the remote computing device;
update the graphical user interface at the remote computing device to enable a virtual display associated with the wearable device.

4. The wearable device of claim 2, wherein:
the graphical user interface includes a depiction of the wearable device based on image data obtained from one or more image sensors of the remote computing device; and
the remote computing device is configured to provide an indication via the graphical user interface that the proximity event has been detected.

5. The wearable device of claim 2, wherein:
the data includes sensor data obtained from the wearable device; and
the user interface includes one or more representations of the sensor data obtained from the wearable device.

6. The wearable device of claim 2, wherein:
the data includes an evaluation of sensor data generated by the one or more sensors of the wearable device.

7. The wearable device of claim 1, wherein the one or more control circuits include one or more processors configured to:
input at least a portion of the sensor data into one or more machine-learned models configured to generate physiological predictions based at least in part on sensor data;
receive a physiological prediction from the one or more machine-learned models in response to the at least a portion of the sensor data;
generate at least one user notification based at least in part on the physiological prediction;
receive a user confirmation input from the user of the wearable device in association with the physiological prediction; and
modify the one or more machine-learned models based at least in part on the user confirmation input.

8. The wearable device of claim 7, wherein modifying the one or machine-learned models comprises:
generating training data based on the at least a portion of the sensor data and the user confirmation input.

9. The wearable device of claim 8, wherein generating training data comprises:
in response to a first user confirmation input, generating positive training data.

10. The wearable device of claim 9, wherein generating training data comprises:
in response to a second user confirmation input, generating negative training data.

11. The wearable device of claim 7, wherein modifying the one or more machine-learned models comprises:
inputting training data to the one or more machine-learned models;
receiving a first prediction in response to the training data;
determining at least one loss function parameter based at least in part on an evaluation of a loss function in response to the first prediction; and
updating the one or more machine-learned models based at least in part on the at least one loss.

12. The wearable device of claim 1, wherein the wearable device is screenless.

13. The wearable device of claim 12, wherein the wearable device is a wristband.

14. The wearable device of claim 1, wherein the one or more sensors include an electrodermal activity (EDA) sensor configured to provide an EDA signal in response to contact between an electrode and a skin surface of a user.

15. The wearable device of claim 1, wherein the one or more sensors include an electrode photoplethysmogram (PPG) sensor configured to provide a PPG signal in response to contact between an electrode and a skin surface of a user.

16. The wearable device of claim 1, further comprising.
one or more non-transitory computer-readable media that collectively store one or more machine-learned models configured to generate physiological predictions based at least in part on the data associated with the physiological characteristics of the user.

17. A user computing device, comprising:
one or more processors; and
one or more non-transitory, computer-readable media that store instructions that when executed by the one or more processors cause the one or more processors to perform operations, the operations comprising:
determining that a proximity event has occurred between the user computing device and a wearable device including one or more sensors configured to generate data associated with one or more physiological characteristics of a user of the wearable device;
receiving, in response to determining that the proximity event has occurred, the data associated with the one or more physiological characteristics of the user;
establishing a virtual display connection between the user computing device and the wearable computing device; and
generating display data for a graphical user interface including a virtual display associated with the wearable device at the user computing device.

18. A wearable device, comprising:
one or more sensors configured to generate sensor data associated with a user;
one or more processors;
one or more non-transitory, computer-readable media that store instructions that when executed by the one or more processors cause the one or more processors to perform operations, the operations comprising:
obtaining the sensor data;
inputting at least a portion of the sensor data into one or more machine-learned models configured to generate physiological predictions;
receiving data indicative of a first physiological prediction from the one or more machine-learned models in response to the at least a portion of the sensor data;
generating at least one user notification based at least in part on the physiological prediction;
receiving a user confirmation input from the user of the wearable device in association with the physiological prediction; and
modifying the one or more machine-learned models based at least in part on the user confirmation input.

19. The wearable device of claim 18, wherein the operations further comprise:
generating training data based on the at least a portion of the sensor data and the user confirmation input.

20. The wearable device of claim 19, wherein modifying the one or more machine-learned models comprises:
inputting the training data to the one or more machine-learned models;
receiving a first prediction in response to the training data;
determining at least one loss function parameter based at least in part on an evaluation of a loss function in response to the first prediction; and
updating the one or more machine-learned models based at least in part on the at least one loss function parameters.
